(19) United States
(12) Patent Application Publication
(10) Pub. No.: US 2004/0014196 A1
(43) Pub. Date: Jan. 22, 2004

(54) Biological anaerobic treatment of BTEX contamination

(75) Inventors: John D. Coates, Walnut Creek, CA (US); Romy Chakraborty, Berkeley, CA (US)

Correspondence Address: FULBRIGHT & JAWORSKI L.L.P., SUITE 2400, 600 CONGRESS AVENUE, AUSTIN, TX, 78701-3271, US

(73) Assignee: Southern Illinois University

(21) Appl. No.: 10/372,628

(22) Filed: Feb. 21, 2003

Related U.S. Application Data
(60) Provisional application No. 60/358,665, filed on Feb. 21, 2002.

(51) Int. Cl. C12S 1/00; C12N 1/20

(52) U.S. Cl. 435/252.1; 435/262.5

(57) ABSTRACT

The present invention concerns methods for isolating microorganisms that can anaerobically degrade aromatic hydrocarbons. The present invention also concerns methods of anaerobic biodegradation of aromatic hydrocarbons.

This patent application claims priority to, and incorporates by reference, U.S. provisional patent application Serial No. 60/358,665 filed on Feb. 21, 2002 entitled, “Biological Anaerobic Treatment of BTEX Contamination.”

The government owns rights in the present invention pursuant to grant number N00014-99-10371 from the US Office of Naval Research and grant number DACA72-00-C-0016 from the US Department of Defense.

### BACKGROUND OF THE INVENTION

1. Field of the Invention

The present invention relates generally to the fields of microbiology. More particularly, it concerns biodegradation of aromatic hydrocarbons.

2. Description of Related Art

Benzene, toluene, ethylbenzene and xylene (BTEX) are prevalent organic contaminants in groundwaters, soil, sediments and aquifers. In particular, benzene is of major concern owing to its toxicity and carcinogenecity. It is insoluble in water but miscible in all proportions with organic solvents. Numerous benzene-degrading aerobic microorganisms have been identified, the most notable of which are the Pseudomonas species, which may account for up to 87% of the petrol-degrading microorganisms in contaminated aquifers (Ridgeway et al, 1990).

However, soils and sediments contaminated with benzene frequently develop extensive anaerobic zones (Anderson et al., 1997; Christensen et al., 1994; Lovley et al., 1997). As a result, the anaerobic biodegradation of benzene under various conditions has been documented. In the last decade, studies have demonstrated that the hydrocarbon-degrading capacity of anaerobes is far greater than previously assumed (Lovley et al., 1994; Lovley et al., 1995; Coates et al., 1996a; Coates et al., 1996b; Coates et al., 1997). Pure cultures of anaerobic organisms that can degrade hydrocarbons such as toluene, hexadecane and naphthalene have been described (Galushko et al., 1999; Rabus et al., 1993; Lovley et al., 1990; Coates et al, 1996). However, organisms capable of anaerobic benzene degradation have been elusive and such organisms have been observed only in sediment studies (Lovley et al., 1994; 1995; 1996; Anderson et al., 1998; 1999; Weiner et al., 1998; Coates et al., 1996; 1997) or in microbial enrichments (Burland et al., 1999; Weiner et al., 1998; Grbic-Galic et al., 1987; Vogel et al., 1986). Organisms capable of anaerobic benzene degradation have not been isolated. Furthermore, even though anaerobic benzene degradation has been demonstrated under various conditions in the absence of O₂ (Burland et al. 1999; Coates et al., 1996; 1997; Grbic-Galic et al., 1987; Vogel et al., 1986; Lovley et al., 1994; 1996), no specific organisms or genera had been associated with the metabolism.

Thus, there remains a need in the art to identify and isolate microorganisms that can anaerobically degrade harmful and recalcitrant aromatic hydrocarbons such as BTEX. There is also a need for a method of biodegrading aromatic hydrocarbons present in anaerobic environments.

### SUMMARY OF THE INVENTION

Thus, in accordance with the present invention, there is provided a method for the biodegradation of an aromatic hydrocarbon, such as benzene, toluene, xylene, or ethylbenzene, comprising the steps of providing a bacterium of the Dechloromonas species, such as strains RCB or JJ, that biodegrades the aromatic hydrocarbon under anaerobic conditions, contacting an environment, such as sediment, soil, groundwater, industrial waste streams or aquifers, containing the aromatic hydrocarbon with the bacterium in the presence of an electron acceptor, such as oxygen, nitrate, chlorate, perchlorate or manganese, and/or further in the presence of trace amounts of ferrous iron or acetate, and incubating the bacterium in the environment for a sufficient time to biodegrade the aromatic hydrocarbon. The trace amounts of ferrous iron and acetate may be at a concentration of 0.1 mM and 1 mM respectively. The incubation may be conducted under aerobic or anaerobic conditions.

In the present embodiment of the invention there is also provided a method for the biodegradation of benzene comprising the steps of providing a bacterium that biodegrades benzene under anaerobic conditions, contacting an environment containing the aromatic hydrocarbon with the bacterium in the presence of an electron acceptor and incubating the bacterium in the environment for a sufficient time to biodegrade the aromatic hydrocarbon. The incubation may be conducted under anaerobic or aerobic conditions. The electron acceptor may be oxygen, nitrate, perchlorate, chlorate, manganese. The environment may be sediment, soil, groundwater, industrial waste streams or aquifers. The bacterial strain may be RCB or JJ of the Dechloromonas sp.

In yet another embodiment, there is provided an isolated bacterium that biodegrades benzene under anaerobic conditions, wherein the bacterium is of the Dechloromonas species. The bacterial strains may be RCB or JJ.

In yet another embodiment of the invention, there is provided a method of isolation of a bacterium that biodegrades one or more aromatic hydrocarbons under anaerobic conditions comprising obtaining a sample containing chlorate- and/or perchlorate-reducing bacteria, incubating the bacteria from the sample in an anoxic medium comprising an electron acceptor and an electron donor; selecting bacteria based on increased cell density in the sample; and inoculating the selected bacteria onto fresh anoxic medium comprising an electron acceptor and an electron donor, wherein the bacteria growing in anoxic medium biodegrades aromatic hydrocarbons under anaerobic conditions. The above steps may be repeated at least two or five or ten times. The electron acceptor may be (per)chlorate, oxygen, nitrate or manganese present at a concentration of 1 mM-25 mM. In a particular embodiment of the invention the electron acceptor, chlorate, may be present at 10 mM. The electron donor may be H₂, volatile fatty acids such as formate, acetate or butyrate, aromatic hydrocarbons such as 4-chlorobenzoate, benzoate, benzene, toluene, ethylbenzene or xylene, reduced metals such as ferrous iron, reduced humic substances or analogs of reduced humic substances such as 2, 6-anthrahydroquinone disulphonate. The electron donor may be present at a concentration of 0.08 mM-20 mM. In a particular embodiment of the invention, chlorate may be present at 0.5 mM. The bacterium may degrade one or more aromatic hydrocarbons selected from the group consisting of benzene, xylene, ethylbenzene, hexadecane, napthalene or toluene. The method may further comprise subjecting the bacterium to an anoxic agar dilution series. The method further comprises incubating and selecting bacteria at in the range of 20-40° C. In a particular embodiment of the invention, the bacteria may be isolated at 30° C.

In a further embodiment, there is provided an isolated bacterium that biodegrades one or more aromatic hydrocarbons under anaerobic conditions, isolated according to the method comprising obtaining a sample containing (per)chlorate-reducing bacteria; incubating the bacteria from the sample in an anoxic medium comprising an electron acceptor and an electron donor; selecting bacteria based on increased cell density in the sample; and inoculating the selected bacteria onto fresh anoxic medium comprising an electon acceptor and an electron donor, wherein bacteria growing on the above mentioned conditions comprise a bacterium that biodegrades aromatic hydrocarbons under anaerobic conditions.

In yet another embodiment of the invention, there is provided a method of reusing an isolated bacterium that biodegrades one or more aromatic hydrocarbons, such as benzene, toluene, xylene, or ethylbenzene, under anaerobic conditions comprising the steps of obtaining bacterial cells from a media that has been depleted of aromatic hydrocarbons; adding a cobalt-containing compound, such as vitamin B₁₂, to these cells and transferring the treated cells to a media containing aromatic hydrocarbons. Vitamin B₁₂ may be added at a concentration of 1 mg/liter. The media from which these bacterial cells are obtained may be either partially depleted of benzene or completely depleted of benzene. The treated bacterial cells may be transferred to a media the same media as above that is partially depleted of aromatic hydrocarbons or it may be transferred to a fresh environment containing aromatic hydrocarbons such as sediment, soil, groundwater, industrial waste stream or aquifer. The media may contain an electron acceptor such as (per)chlorate, oxygen, nitrate or manganese. The bacterium used may be strains RCB and/or JJ of Dechloromonas sp.

The present embodiment of the invention further provides for a method of enhancing the efficiency of degradation of aromatic hydrocarbons in a media comprising adding a cobalt-containing compound, such as vitamin B₁₂, to the media. The vitamin B₁₂ may be added after the initiation of benzene oxidation. In particular embodiment of the invention, vitamin B₁₂ may be added 24 hours after the initiation of benzene oxidation. The media may be sediment, soil, groundwater, industrial waste stream or aquifer. The degradation may be carried out by strains of Dechloromonas sp such as RCB or JJ.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Scanning electron micrograph of anaerobically grown cells of strain RCB. The horizontal bar represents 0.2 μm.

FIG. 2—Growth of strain RCB with 2,6-anthrahydroquinone disulfonate (5 mM) as the electron donor and chlorate (10 mM) as the electron acceptor. Acetate (0.1 mM) was added as a suitable carbon source. Closed circles: AHDS concentration in growth culture; open circles: AHDS concentration in heat killed controls; closed triangles: cell density in growth cultures: open triangles: cell density in the heat killed controls. The results depicted are the averages of triplicate determinations.

FIG. 3—Phylogenetic tree of the 16S rDNA sequences of strains RCB and JJ and their closest relatives resulting from a heuristic search using parsimony analysis. The same topology was obtained from distance and maximum likelihood, and was supported by bootstrap analysis.

FIGS. 4A-4C—Anaerobic Oxidation of Benzene by JJ and RCB FIG. 4A: [¹⁴C]-Benzene is completely oxidized to ¹⁴CO₂ by strain RCB (closed circles) and strain JJ (closed squares) in the absence of oxygen with nitrate as the electron acceptor. No ¹⁴CO₂ production was observed with either strain RCB (open circles) or strain JJ (open squares) in the absence of nitrate or if the cells were heat killed. FIG. 4B: Anaerobic growth and benzene oxidation by strain RCB with nitrate as the electron acceptor. Closed squares: benzene concentration in the growth culture; open squares: benzene concentration in the heat killed controls; closed circles: cell numbers in the growth culture; open circles: cell numbers in the absence of benzene. FIG. 4C: Growth and benzene oxidation, in fresh anoxic culture media, with nitrate as the electron acceptor after inoculation (10% vol/vol) from the active growth culture tubes from b above. Closed squares: benzene concentration in the growth culture; open squares: benzene concentration in the heat killed controls; closed circles: cell numbers in the growth culture; open circles: cell numbers in the absence of benzene.

FIG. 5—[¹⁴C]-Benzene degradation in anoxic sediments amended with nitrate (10 mM) in the presence and absence of strain RCB. The results depicted are the averages of triplicate determinations.

FIGS. 6A-6C—Effect of some compounds on the oxidation of benzene. FIG. 6A: Effect of vitamin B₁₂ on the oxidation of benzene. FIG. 6B: Effect of propyl iodide on oxidation of benzene. FIG. 6C: Effect of benzene and toluene on the oxidation of benzene. The results depicted are the averages of triplicate determinations.

FIGS. 7A-7B—Results of field trial. FIG. 7A: Nitrate concentration in test plot and control plot soils during field trial. FIG. 7B: Benzene concentration in test plot and control plot soils during field trial.

FIGS. 8A-8B—Results of Trial A. FIG. 8A: Benzene removal in aerobic soils over time. FIG. 8B: Benzene removal in anaerobic soils over time.

FIG. 9—Benzene removal with time in anaerobic samples from Trial B.

FIGS. 10—¹⁴CO₂ production from [¹⁴C]-benzene by strain RCB with oxygen, nitrate, or chlorate as electron acceptors. The results depicted are the averages of triplicate determinations.

FIG. 11—Typical HPLC chromatogram obtained from analysis of culture broth from an anoxic [¹⁴C]-benzene-degrading culture of permeabilized cells of strain RCB amended with unlabeled toluene (35 μM). The chromatogram indicates the presence of several [¹⁴C]-labeled compounds including [¹⁴C]-toluene.

FIG. 12—Inhibition of ¹⁴CO₂ production from [¹⁴C]-benzene by addition of toluene, benzylalcohol, or benzaldehyde to anaerobic cultures of strain RCB. The results depicted are the averages of triplicate determinations.

### DESCRIPTION OF ILLUSTRATIVE EMBODIMENTS

Aromatic hydrocarbon contamination of soil, sediments, groundwater and aquifers remains a significant problem. Aromatic hydrocarbons such as benzene, toluene, ethylbenzene and xylene (BTEX) are widely used in various manufacturing processes and are also a primary component of petroleum-based fuels.

In as much as aromatic hydrocarbons form a very important basis of various compounds utilized in our day to day activities, they are unavoidable environmental contaminants. Unfortunately, they can have very harmful effects, especially if remaining undegraded. This is especially true of benzene due to the high solubility and carcinogenecity of this compound.

One of the most important ways by which a person is exposed to the harmful effects of aromatic hydrocarbons is by exposure to the effluents from industrial processes. Industrial processes are the main source of benzene in the environment. Benzene can pass into the air from water and soil. It reacts with other chemicals in the air and breaks down within a few days. Benzene in the air can attach to rain or snow and be carried back down to the ground. It breaks down more slowly in water and soil, and can pass through the soil into underground water (Agency for Toxic Substances and Disease Registry, website: www.atsdr.cdc.gov). Among the diseases that have been associated in the literature with benzene are acute myelogenous leukemia (AML), acute lymphocytic leukemia (ALL), chronic myelogenous leukemia (CML), chronic lymphocytic leukemia (CLL), hairy cell leukemia (HCL), non-Hodgkin's lymphoma (NHL), Hodgkin's disease, multiple myeloma, myelodysplastic syndrome (MDS), aplastic anemia, pancytopenia, other cytopenias, myelofibrosis, and polycythemia vera. Recent studies from China and Great Britain establish that benzene can cause various hematologic cancers and blood diseases at extraordinarily low doses a few ppmy (part per million years). A part per million year (ppmy) of benzene is a cumulative dose of just 1 ppm, which is permissible under the Occupational Safety and Health Administration (OSHA)'s allowed exposure limit for benzene.

There is a need in the art for aromatic hydrocarbons to be degraded especially in soils which develop anaerobic and anoxic zones. Bioremediation is a cost effective and natural treatment process, applicable to many organic wastes, that uses naturally occurring microorganisms (yeast, fungi, or bacteria) to breakdown or degrade hazardous substances into less toxic or nontoxic substances. Although bioremediation holds great promise for dealing with intractable environmental problems, it is important to recognize that much of this promise has yet to be realized.

Various aerobic microorganisms are able to degrade the aromatic hydrocarbons under aerobic conditions, but a large number of aromatic hydrocarbons, predominantly, BTEX, remain trapped and undegraded in soil and sediments. This is because various aerobic microorganisms utilize the existing amounts of oxygen to degrade BTEX, slowly depleting the reserves of oxygen in the soil. This, in turn, leads to the formation of anaerobic zones in the soil that results in the inability of these aerobes to further degrade the compounds. In particular, benzene is poorly biodegraded anaerobically. However, recent studies have documented anaerobic benzene biodegradation under various conditions. Although benzene biomineralization has been demonstrated with nitrate (Burland et al., 1999), Fe(III) (Lovley et al., 1994; 1996; Anderson et al., 1998; 1999), sulfate (Lovley et al., 1995), CO₂ (Weiner et al., 1998; Grbic-Galic et al., 1987), as alternative electron acceptors, all of these studies were based on sediments or microbial enrichments. Until now, there were no organisms in pure culture that degraded benzene anaerobically.

I. The Present Invention

The present invention provides microbial strains of Dechloromonas species such as JJ and RCB strains, that can be isolated and maintained as a pure culture and can efficiently degrade environmental contaminants such as BTEX from anaerobic environments such as industrial wastes, sediments, soil, groundwater or aquifers. The present invention also provides a methodology for the isolation of such organisms. The Dechloromonas strains of the present invention can oxidize aromatic hydrocarbons anaerobically with the use of electron acceptors such as oxygen (O₂), nitrate (NO₃¹⁻), manganese (Mn⁴⁺), perchlorate (ClO₄¹⁻), and chlorate(ClO₃¹⁻). The microorganisms of the present invention may be used as an effective tool for bioremediation of anaerobic environments contaminated with aromatic hydrocarbons.

II. Aromatic Hydrocarbons

Aromatic Hydrocarbons are hydrocarbons based on the benzene ring as a structural unit. A few examples of aromatic hydrocarbons are benzene, toluene, ethylbenzene and xylene.

Benzene. Benzene was discovered in 1825 by the English scientist Michael Faraday. Benzene is a colorless liquid with a characteristic odor and burning taste and has the formula C₆H₆. The benzene molecule is a closed ring of six carbon atoms connected by bonds that resonate between single and double bonds; each carbon atom is also bound to a single hydrogen atom. The resonance energy makes the benzene a very stable compound. It is insoluble in water, but miscible in all proportions with organic solvents. Benzene itself is an excellent solvent for certain elements, such as sulfur, phosphorus, and iodine; for gums, fats, waxes, and resins; and for most simple organic chemicals. It is one of the most commonly used solvents in the organic chemical laboratory.

Benzene melts at 5.5° C. (41.9° F.), boils at 80.1° C. (176.2° F.), and has a specific gravity of 0.88 at 20° C. (68° F.). If inhaled in large quantities, benzene is poisonous. The vapors are explosive, and the liquid violently flamnable. Many compounds, such as nitrobenzene, are obtained from benzene. Benzene is also used in the manufacture of drugs and in the production of important derivatives, such as aniline and phenol. Benzene and its derivatives are included in the important chemical group known as aromatic compounds. They are used in manufacture of plastics, nylon, synthetic fibres, some types of rubbers, lubricants, dyes, detergents, drugs and pesticides.

Benzene as an organic chemical is used as an additive in gasoline and as a raw material for the production of hundreds of other chemicals. Over 12 billion pounds are produced annually. It is emitted to the air from five principal sources: (1) auto emissions (2) gasoline evaporation (3) benzene production (4) benzene consumption and (5) coke oven operations.

When mixed with a large proportion of gasoline it makes a satisfactory fuel. In Europe benzene mixed with some toluene and other related compounds has long been added to motor fuels.

Toluene. Toluene is simply benzene with an extra methyl group added to the ring, and is well-known as the precursor to trinitrotoluene (TNT). It got its name because it was originally obtained from the gum of the South American tree Toluifera balsamum. This balsum, commonly called Tolu balsum, is yellowy-brown with a pleasant aroma, and has been used in perfumes and cough syrup.

Toluene (methylbenzene) is an aromatic hydrocarbon natural product of diagenic origin and an important commercial chemical. It is, for example, commonly used as a paint-thinning agent and in other solvent applications. The biodegradation of toluene has been well studied at the molecular level and it, thus, serves as one of the principal models for understanding the mechanisms of bacterial benzene ring metabolism.

Ethylbenzene. Ethylbenzene is a colorless liquid that melts like gasoline. It evaporates at room temperature and burns easily. Ethylbenzene occurs naturally in coal tar and petroleum. It is also found in many man-made products, including paints, inks, and insecticides. Gasoline contains about 2 percent (by weight) ethylbenzene. Ethylbenzene is commonly found as a vapor in the air because ethylbenzene moves easily into the air from water and soil. Once in the air, other chemicals help breakdown ethylbenzene into chemicals found in smog. Ethylbenzene can move very quickly in groundwater, since it does not bind readily to soil.

Xylene. The xylenes (from the Greek xulon for wood, since they were once obtained by distilling wood in the absence of air), have 2 methyl groups attached to the ring. There are 3 possible isomers of xylene, ortho-xylene where the two methyls are next to each other, meta-xylene where they are separated by a hydrogen atom, and para-xylene where they are on opposite sides of the ring. A mixture of the 3 isomers is called xylol and is often used as a solvent. Both toluene and xylene occur in gasoline (along with benzene). Their concentrations can be deliberately increased during the refining process to give a high performance (Benzene Toluene Xylene) BTX gasoline used for sports cars.

III. Biodegradation of Aromatic Hydrocarbons

Biodegradation is defined as the transformation of a chemical that results in a change of composition, state, and/or chemical properties.

The most effective way to remediate organic contamination is biodegradation. Biodegradation is useful for many types of organic wastes and is a cost-effective, natural process. Many techniques can be conducted on-site, eliminating the need to transport hazardous materials.

The extent of biodegradation is highly dependent on the toxicity and initial concentrations of the contaminants, their biodegradability, the properties of the contaminated soil, and the particular treatment system selected. Contaminants targeted for biodegradation treatment are volatile and semi-volatile organics and fuels. The effectiveness of bioremediation is limited at sites with high concentrations of metals, highly chlorinated organics, or inorganic salts because these compounds may be toxic to microorganisms.

The present invention concerns the anaerobic biodegradation of aromatic hydrocarbons such as benzene. As mentioned earlier in the application, the resonance energy makes the benzene a very stable compound and hence it is recalcitrant to biodegradation.

A. Anaerobic Biodegradation

Oxidation of aromatic hydrocarbons, such as benzene, toluene, ethylbenzene, and xylenes (BTEX) by anaerobic bacteria is of considerable biochemical and environmental interest. The enzymes involved in the initial degradation reactions of these substrates are dramatically different from the well-characterized aerobic oxidation reactions. In all aerobic pathways, the initial reactions involve mono- or dioxygenases that require molecular oxygen as a substrate. Anaerobic bacteria face the biochemical problem of how to perform the first oxidation step in the absence of molecular oxygen.

At some contaminated sites described earlier, as a result of consumption of O₂ by aerobic microorganisms and slow recharge of O₂, the environment becomes anaerobic (lacking O₂), and mineralization, transformation, and co-metabolism depend upon microbial utilization of electron acceptors other than O₂ (anaerobic biodegradation). Nitrate (NO₃), iron (Fe⁺³), manganese (Mn⁴⁺), sulfate (SO₄), perchlorate (ClO₄¹⁻), chlorate (ClO₃¹⁻) and carbon dioxide (CO₂) can act as electron acceptors if the organisms present have the appropriate enzymes. The Dechloromonas species of the present invention may be used to anaerobically degrade benzene in such anaerobic pockets.

B. Microorganisms Involved in Degradation

Biodegradation is facilitated by bacteria. The activity of these bacteria is controlled by the concentration of contaminants and by the presence of electron acceptors, which allows the bacteria to oxidize or reduce the contaminants.

Microorganisms decompose organic compounds using enzymes that control metabolism in all living cells. Some of these enzymes act to break down, or biodegrade, pollutants, and this natural process can be exploited for the treatment of industrial, agricultural, or municipal wastes. Different microorganisms may be used to perform different stages of a degradation pathway.

Dechloromonas are rod-shaped, gram-negative cells 0.5 by 2 μm, non-sporeforming, non-fermenting, facultative anaerobe. Cells are motile by a single polar flagellum and occur singly or in chains of 2 to 3 cells. Compounds such as chlorobenzoate or acetate serve as electron donors and oxygen, nitrate, perchlorate, chlorate and manganese can serve as electron acceptors for the growth and metabolism of this organism.

All the Dechloromonas species described to date are heterotrophic facultative anaerobic respirers (Bruce, 1999; Coates et al., 1999b; Michaelidou et al., 2000). Some of the isolates can alternatively use nitrate as an electron acceptor (Coates et al., 1999b) and nitrate is completely reduced to N₂.

Dechloromonas strain RCB and Dechloromonas strain JJ are closely related to each other and both are members of the Dechloromonas subgroup in the β subclass of the Proteobacteria. Their closest relative is Ferribacterium limneticum (Coates et al., 2001a). They can completely mineralize various monoaromatic compounds including benzene, toluene, ethylbenzene and xylene to CO₂ in the absence of O₂ with electron acceptors listed earlier in the application. Owing to the ubiquity of the Dechloromonas genus (Coates et al., 1999) they may be isolated from diverse environments.

IV. Methods of Isolating Bacterial Anaerobic Biodegraders of Aromatic Hydrocarbons

Many specific kinds of microorganisms can be obtained from their natural habitats such as soil and water by the creation in the laboratory of an artificial environment which will enhance their growth over competing organisms. Morphological and/or physiological characteristics of the desired organisms which can give them selective advantages over others are exploited in the formulation of culture media, the choice of incubation conditions, and any special treatment of the original source material itself. It is desired that as many “undesirable” organisms as possible are inhibited so that they do not interfere with the isolations of the desired organisms, and it is also desired that the nutritional requirements of the desired organisms be satisfied such that they grow well.

To help detect and isolate a certain kind of organism and minimize interference by other organisms, the following considerations are made: choice of suitable source material likely to contain the desired organism; choice of whether or not any special treatment of the source material is needed. The more “selection” that is done at this initial stage of the isolation process, the less selective the subsequent isolation medium needs to be; choice of whether to plate the sample directly or subject it to an enrichment.

Often the source material is inoculated directly into a defined medium which will encourage the proliferation of the desired organism; this is called an enrichment.

Suitable formulation of the isolation medium may be carried out. Isolation medium is a plating medium upon which one obtains isolated colonies, having practiced this technique numerous times. Usually, a selective medium is employed. In general, selection may be achieved by either adding a selective agent to poison undesired organisms (as in MacConkey Agar) or making the medium restrictive by including a nutrient only certain organisms can use, or by leaving something out. Utilization of suitable incubation conditions such as suitable temperature, amount of oxygen, light.

The detection of desired organisms by making use of the property of microbial groups that have recognizable cultural and/or morphological characteristics which aid in their detection, some may be detected simply as a result of outgrowing others.

Methods for isolating bacteria that biodegrade aromatic hydrocarbons under anaerobic conditions generally consist of the following steps: obtaining a bacterial sample from a suitable source; incubating the bacteria from the sample in an anoxic media in the presence of an electron donor and acceptor; selecting the bacteria based on characteristics such as increased cell density; inoculating the selected bacteria onto fresh anoxic medium in the presence of an electron donor and acceptor. The last two steps namely, selecting a bacteria and inoculating in a fresh anoxic medium may be repeated several times.

A. Isolation Methodology for strains of Dechloromonas Species

Strains of Dechloromonas species may be enriched with oxygen, perchlorate, chlorate (collectively known as (per)chlorate) or nitrate as suitable electron acceptors and various alternative electron donors including H₂, volatile fatty acids (formate, acetate, butyrate), aromatic compounds (chlorobenzoate, benzoate, benzene, toluene, ethylbenzene and xylene), reduced metals (Fe(II)), or reduced humic substances or analogs thereof (e.g., 2,6-anthrahydroquinone disulfonate). Dechloromonas strain RCB was enriched and isolated as a 4-chlorobenzoate-oxidizing chlorate reducer, while Dechloromonas strain JJ was isolated as a humic substance-oxidizing nitrate-reducer. Enrichments may be initiated in a temperature range of 20° C. to 40° C. in anoxic artificial medium with an electron donor and an electron acceptor as outlined above. A concentration of 0.08 mM to 20 mM of the electron donor and a concentration of 1 mM to 25 mM of the electron acceptor may be used depending on the compounds selected.

Inocula may be collected from a source previously demonstrated to contain an active microbial population such as industrial wastes, farm animal wastes, sediments, soil, groundwater or aquifers such as petroleum contaminated aquifers. Positive enrichments may be identified by microscopic observation of cell density increase over time, change in color of media or transformation of the electron donor or acceptor used. After the cell numbers increase by at least one log unit, the cultures may be transferred. It is contemplated that the amount of culture transferred vol/vol may be at least 0.1% to at most 10% into freshly prepared media. After several transfers in electron acceptor/electron donor medium the enrichments may be inoculated into an anoxic agar dilution series (Bruce et al., 1999; Coates et al., 1999a) based on the same medium amended with 2% noble agar. The present invention contemplates that the number of transfers of the culture in fresh media may be repeated at least once, at least five times or at least ten times. The number of transfers is not a limiting factor. The higher number of transfers leads to a higher degree of selection. Colonies of strains of Dechloromonas generally appear within three to six weeks as small white/orange colonies 1-2 mm in diameter.

B. Detection and characterization of the Desired Organism During the Enrichment and Isolation Process

Many organisms have characteristics associated with them that aid in their detection during enrichment or isolation. Some may show pigmentation, some may have peculiar cell shapes and some may form endospores. Bergey's Manual allows one to identify dozens of species of various genera.

V. Detection and measurement of Aromatic Hydrocarbon Degradation

The following section highlights the various methods by which the degradation of aromatic hydrocarbons may be measured or quantified. One of the ways by which it may be done is to collect samples from the biodegradation reaction mixture at various time points during the degradation process and measure it for the amount of aromatic hydrocarbons that remain undegraded or the amount and types of byproducts that are produced as a result of biodegradation. There are various techniques that may be used such as gas chromatography (GC), mass spectrometry (MS), High Performance Liquid Chromatography (HPLC) and Liquid Scintillation Counting (LSC).

A. Gas Chromatography (GC)

Gas Chromatography is a technique for separating mixtures which relies upon the differential distribution of their components between a stationary, condensed phase and a mobile, gaseous phase. A measured volume of the sample is injected into an analytical column through which it is carried by an inert carrier gas. Adsorption or partitioning agents in the column retard the various components of the sample by varying degrees so that they emerge from the column at different times. Only a fraction of the mixture is delivered into the column, the remainder is vented to the atmosphere. The separated components pass through a detector which gives an electrical signal that depends on their concentration in the carrier gas. Most frequently used carrier gas is helium (He).

B. Mass Spectrometry (MS)

A mass spectrometer is an apparatus that converts molecules into ions and then separates the ions according to their mass-to-charge ratio. Mass spectrometers are used to identify atoms and isotopes, and determine the chemical composition of a sample. All mass spectrometers have four features in common: (1) a system for introducing the substance to be analyzed into the instrument; (2) a system for ionizing the substance; (3) an accelerator that directs the ions into the measuring apparatus; and (4) a system for separating the constituent ions and recording the mass spectrum of the substance. Mass spectrometers can provide a high degree of resolution to aid in the analysis of complex mixtures. Products of petroleum refining and processing, for example, which usually contain various closely related hydrocarbons, are difficult to separate by conventional methods of chemical analysis, but can be isolated and analyzed using a mass spectrometer.

C. Liquid Scintillation Counting (LSC)

Liquid Scintillation Counters have become the most efficient and versatile detection systems for beta-emitting radioisotopes. Fluorescence provides the basis for liquid scintillation counting. To maximize the fluorescence yield, the radioisotope is placed in intimate contact with a solution of the detector molecules, which are fluorescent molecules (fluorophores). The solvent used in these solutions, suspensions or emulsions is very important since it must participate in the energy transfer to the fluorophore molecules. The energy transfer occurs very rapidly (less than 10-8 sec) and the fluorophores upon activation and decay yield a flash of visible light (photons). Not all of the beta particles have sufficient energy to cause sufficient photon production to activate the photomultiplier tubes (a pair of tubes is used) of the counter.

D. High Performance Liquid Chromatography (HPLC)

High Performance Liquid Chromatography is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

E. Others

Any compound that is a substrate for microorganisms can be used to estimate their activity if the decrease in substrate concentration can be measured. In the present invention the rate of benzene degradation may be measured and used as a method to determine the activity and efficiency of Dechloromonas in degrading benzene. Similarly, the rate of product accumulation may also be used as a measure of efficient conversion of a compound such as accumulation of CO₂ as a result of benzene degradation in the present invention.

Colorimetric methods, as used in the present invention for measuring the AUDS concentrations, may be adopted.

Radioisotope cycling studies may also be carried out as described. Many elements can be obtained in the form of a radioactive isotope such as ¹⁴C, ³²p and ³H. They can be used to measure the activities of microorganisms growing and metabolizing compounds that include these isotopes. For example, benzene can be labeled with ¹⁴C in specific positions in the benzene molecule and the fate of the radiolabeled atom can be traced through standard chemical analysis followed by detection of the radioactivity in specific fractions. If the radiolabeled atom has been evolved as CO₂ through respiration of the cells, it will make CO₂ radioactive and this can be detected.

VI. Industrial Bioremediation

Bioremediation is a treatment process that uses naturally-occurring microorganisms (yeast, fungi, or bacteria) to breakdown, or degrade, hazardous substances into less toxic or nontoxic substances. Once the contaminants are degraded, the microorganism population is reduced because they have utilized their entire food source. Dead microorganisms or small populations in the absence of food pose no contamination risk.

Due to its comparatively low cost and generally benign environmental impact, bioremediation offers an attractive alternative and/or supplement to more conventional clean-up technologies. Bioremediation has been successful at many sites contaminated with petroleum products.

Bioremediation works best on natural hydrocarbons or on chemicals resembling natural substances. Bacteria that metabolize naturally-occurring hydrocarbons, such as certain petroleum products, are widespread in the environment. Certain bacteria that biodegrade gasoline, for example, are found in almost all soils; the gasoline-metabolizing bacteria may be isolated from the other bacteria present in the soil by laboratory testing. Some of the bacteria employed in bioremediation include members of the genera Pseudomonas, Dechloromonas, Flavobacterium, Arthrobacter, and Azotobacter. Some toxic substances that have been successfully bioremediated in the past include the solvent toluene the moth repellent naphthalene (mothballs), the herbicide 2,4-dichlorophenoxyacetic acid (2,4-D), and the fungicide and wood preservative pentachlorophenol.

In the present invention the use of Dechloromonas for the bioremediation of aromatic hydrocarbons from anaerobic zones in soil and sediments is contemplated. It represents a treatment technology in its own right.

Bioremediation technologies assist microorganisms' growth and increase microbial populations by creating optimum environmental conditions for them to detoxify the maximum amount of contaminants. The specific bioremediation technology used is determined by several factors, for instance, the type of microorganisms present, the site conditions, and the quantity and toxicity of contaminant chemicals. Different microorganisms degrade different types of compounds and survive under different conditions.

If the biological activity needed to degrade a particular contaminant is not present in the soil at the site, microorganisms from other locations, whose effectiveness has been tested, can be added to the contaminated soil. These are called exogenous microorganisms. The soil conditions at the new site may need to be adjusted to ensure that the exogenous microorganisms will thrive. In the present invention, it is contemplated that the Dechloromonas species act as exogenous microorganisms. As mentioned earlier in the application, the organism uses electron donors such as oxygen, (per)chlorate, nitrate, and manganese. It has also been observed that the use of an electron acceptor such as chlorate in combination with trace amounts of ferrous iron or acetate significantly enhances the anaerobic benzene oxidation. The ferrous iron and acetate may be used at a concentration of 0.1 mM and 1 mM respectively.

In environments where there is a presence of Dechloromonas species they may act as endogenous microorganisms. Under conditions where they lack a limiting nutrient such as nitrate, an external source of nitrate may be provided to them.

Bioremediation can take place under aerobic and anaerobic conditions. These terms are described earlier in the invention. Bioremediation can be used as a cleanup method for contaminated soil, water and wastestreams. Bioremediation applications fall into two broad categories: in situ or ex situ. In situ bioremediation treats the contaminated soil or groundwater in the location in which it was found. Ex situ bioremediation processes require excavation of contaminated soil, extraction of the bound contaminant or pumping of groundwater before they can be treated. The microorganisms of the present invention may be used in carrying out both aerobic and anaerobic in situ or ex situ bioremediation.

A. In situ Bioremediation of Soil

In situ techniques do not require excavation of the contaminated soils so may be less expensive, create less dust, and cause less release of contaminants than ex situ techniques. Also, it is possible to treat a large volume of soil at once.

In situ bioremediation of groundwater speeds the natural biodegradation processes that take place in the water-soaked underground region that lies below the water table. For sites at which both the soil and groundwater are contaminated, this single technology is effective at treating both. It is contemplated that the microorganisms of the present invention would be very useful in such a process. They can also be used to enhance in-situ aerobic bioremediative treatment processes as these organisms can be rapidly grown and will use BTEX compounds both aerobically and anaerobically. Other applications may include treatment of other contaminants, both organic and inorganic including ferrous iron (Fe (II)), sulfide (H₂S), and perchlorate (ClO₄⁻), toxic.

B. Ex situ Bioremediation of Soil

Ex situ techniques can be faster, easier to control, and used to treat a wider range of contaminants and soil types than in situ techniques. However, they require excavation and treatment of the contaminated soil before and, sometimes, after the actual bioremediation step. Ex situ techniques include slurry-phase bioremediation and solid-phase bioremediation. The Dechloromonas species of the present invention have the capability of degrading contaminants both aerobically and anaerobically and the ex situ bioremediative process may be considered for some contaminant samples where in situ bioremediation may not be applicable.

Slurry-phase bioremediation. Contaminated soil is combined with water and other additives in a large tank called a “bioreactor” and mixed to keep the microorganisms, which are already present in the soil, in contact with the contaminants in the soil. Nutrients and an appropriate electron acceptor are added, and conditions in the bioreactor are controlled to create the optimum environment for the microorganisms to degrade the contaminants. Upon completion of the treatment, the water is removed from the solids, which are disposed off or treated further if they still contain pollutants.

Slurry-phase biological treatment can be a relatively rapid process compared to other biological treatment processes, particularly for contaminated clays. The success of the process is highly dependent on the specific soil and chemical properties of the contaminated material. This technology is particularly useful where rapid remediation is a high priority.

Solid-phase bioremediation. Solid-phase bioremediation is a process that treats soils in above-ground treatment areas equipped with collection systems to prevent any contaminant from escaping the treatment. Moisture, heat, nutrients, electron donors or acceptors are controlled to enhance biodegradation for the application of this treatment. Solid-phase systems are relatively simple to operate and maintain, require a large amount of space, and cleanups require more time to complete than with slurry-phase processes. Solid-phase soil treatment processes include landfarming, soil biopiles and composting.

C. The Role of Cobalt-Containing Compounds in Increasing the Efficiency of BTEX Oxidation.

Cobalt-containing compounds, play a significant role in the biomethylation of various heavy metals. Vitamin B₁₂ is a cobalt containing corrinoid generally involved in bioalkylation reactions (Berman et al., 1990; Choi et al., 1994; Ridley et al., 1977; Wood and Wolfe, 1966). It is thought to play a significant role in the biomethylation of various heavy metals (Choi and Bartha, 1993; Fatoki, 1997). The present invention contemplates the use of a cobalt-containing compound such as vitamin B₁₂ to enhance the efficiency of biodegradation of aromatic hydrocarbons such as benzene. Vitamin B₁₂ may be added to the media undergoing degradation of aromatic hydrocarbons. In a particular embodiment of the invention, vitamin B₁₂ may be added after benzene oxidation has been initiated. In a preferred embodiment of the invention, vitamin B₁₂ may be added 24 hours after the initiation of benzene oxidation. Alternatively, vitamin B₁₂ may be added to bacterial cells obtained from a media subjected to aromatic hydrocarbon biodegradation. These bacterial cells may be isolated from the media, washed and treated with a cobalt-containing compound such as vitamin B₁₂. After being treated, the cells may be used for a second round of aromatic hydrocarbon degradation. The addition of vitamin B₁₂ may be very useful in a situation where the degradation of aromatic hydrocarbons is incomplete but has ceased because the organisms have already reached a plateau as far as their rate of degradation of benzene is concerned. Alternatively, cobalt containing compounds may be added early in the degradation reaction such as 24 hours after the initiation of benzene oxidation so that the cells continue to degrade benzene.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Examples 2-7

Media preparation. All media and solutions were prepared using strict anaerobic techniques as previously described (Lovley et al., 1995; Bruce et al., 1999; Coates et al., 1999). All culturing was done in sealed serum vials with N₂-CO₂ (80-20, vol/vol) in the headspace. Basal medium used for the isolation of strain RCB was amended from a sterile aqueous anoxic stock solution of 4-chlorobenzoate (100 mM) to give a final concentration of 0.5 mM when appropriate. Basal medium used for the isolation of strain JJ was prepared from anoxic freshwater medium containing 2,6-anthraquinone disulfonate (AQDS) reduced with palladium coated aluminum chips and H₂ as previously described (Lovley et al, 1999).

In general, alternative electron donors tested were added from sterile aqueous anoxic stock solutions. When necessary, benzene was added from an anoxic aqueous stock solution prepared as previously described (Lovley et al., 1995). Growth was determined by observation of cell density increase and was confirmed by direct cell counts under oil immersion phase contrast microscopy. Results were compared with those of unamended controls. [¹⁴C]-Benzene (1 μCi) was added from an anoxic stock solution as previously described (Coates et al., 1998; 1999).

Isolation Methodology for RCB strains of Dechloromonas species. Strain RCB was enriched as a hydrocarbon-oxidizing chlorate reducer. Enrichments were initiated at 30° C. in anoxic artificial medium (Bruce et al., 1999) with 4-chlorobenzoate (0.5 mM) as the electron donor and chlorate (10 mM) as the electron acceptor. Inocula were taken from sediments collected from Potomac River, Md. previously demonstrated to contain an active microbial population that could reduce either chlorate or perchlorate [(per)chlorate] (Coates et al., 1999a, b) and an anaerobic hydrocarbon-degrading population (JD Coates, unpublished observation). Positive enrichments were identified by microscopic observation of cell density increase over time. After 8 days, cell numbers increased by at least one log unit. The cultures were transferred (10% vol/vol) into freshly prepared media. After several transfers in chlorobenzoate/chlorate medium the enrichments were inoculated into an anoxic agar dilution series (Bruce et al., 1999; Coates et al., 1999a) based on the same medium amended with 2% noble agar. Colonies of strain RCB were isolated after six weeks incubation.

Isolation Methodology for JJ strains of Dechloromon as species. Strain JJ was isolated as a humic substances-oxidizing nitrate-reducer. Enrichments were initiated at 30° C. in anoxic artificial medium (Bruce et al., 1999) with 2,6-anthrahydroquinone disulfonate (AHDS) (5 mM) as the electron donor and nitrate (5 mM) as the electron acceptor with 0.1 mM acetate as a carbon source. AHDS medium was prepared from anoxic freshwater medium containing 2,6-anthraquinone disulfonate (AQDS) reduced with palladium coated aluminum chips and H₂ (Lovley et al., 1999). Inocula were taken from sediments collected from Campus Lake, Southern Illinois University. Positive enrichments were identified by color change in the media (red to tan) as the AHDS was oxidized. The cultures were transferred (10% vol/vol) into freshly prepared anoxic AHDS/nitrate medium twice over three weeks and then inoculated into an anoxic agar dilution series (Bruce et al., 1999; Coates et al., 1999a) based on the same medium amended with 2% noble agar. Colonies of strain JJ were isolated after three weeks incubation.

Sediment experiments. Fresh sediments were collected from Southern Illinois University at Carbondale (SIUC) Campus Lake and stored under N₂ at 4° C. until utilized. Geochemical and terminal electron accepting process analyses were performed directly before use as previously described (Coates et al., 1996a; b; 1997). All experiments were performed in triplicate using strict anaerobic techniques in sealed 35 ml serum vials amended with 5 g sediment under an N₂ headspace. The inoculum size was 10% vol/wt of anaerobically grown cells with nitrate (10 mM) as the sole electron acceptor. [¹⁴C]-Benzene (1 μCi) was added from a stock solution (Lovley et al., 1995) and benzene concentrations were determined by GC-GPC as previously described (Coates et al., 1998; 1999). Nitrate was amended from an anoxic sterile stock solution.

Electron microscopy. Scanning electron micrographs were prepared using cells grown anaerobically with acetate (10 mM) as the electron donor and nitrate (10 mM) as the electron acceptor as previously described (Bruce et al., 1999) and viewed with a Hitachi S570 SEM at 20 kV.

Analytical techniques. Concentrations of ¹⁴CO₂ in the headspace of cultures amended with [¹⁴C]-benzene was determined by gas chromatography with gas proportional counting detection (GC-GPC) as previously described (Coates et al., 1998; 1999). Benzene concentrations in growth cultures were determined by gas chromatography of 0.1 ml headspace samples as previously described (Lovley et al., 1995). Concentrations of aqueous phase benzene and potential catabolic intermediates were determined by high-pressure liquid chromatography (HPLC) using a LC-18 Supelcosil column with UV detection at 254 nm. The mobile phase was 60/40 (vol/vol) methanol:water. If [¹⁴C]-benzene was used, eluted peaks were collected directly into 5 ml of liquid scintillant and analyzed for [¹⁴C]-label by liquid scintillation counting using a Beckman LS 6000IC. Nitrate concentrations were determined by ion chromatography of aqueous samples using a Dionex DX500 equipped with an AS9-SC column using a sodium carbonate (2 mM)/sodium bicarbonate (7.5 mM) mobile phase at a flow rate of 2 ml/min. AHDS concentrations were determined as previously described by calorimetric assay at 450 nm (Lovley et al, 1999). Cell numbers were determined by direct microscopic counting under oil immersion.

Phylogenetic analysis. Polymerase chain reaction (PCR), sequencing and analysis of the 16S rRNA genes was performed as previously described (Coates et al., 1997; Achenbach et al., 2001). Sequence entry and manipulation was performed with the MacVector 6.5 sequence analysis software program for the Macintosh (Oxford Molecular Group). Sequences of select 16S rRNAs were downloaded from the Ribosomal Database Project and Genbank into the computer program SeqApp. 16S rDNA sequences of strains RCB and JJ were manually added to the alignment using secondary structure information for proper alignment (alignment available on request). The Genbank Accession No. for partial sequence of Dechloromonas strains RCB and JJ 16S ribosomal RNA gene are AY032610 and AY032611 respectively.

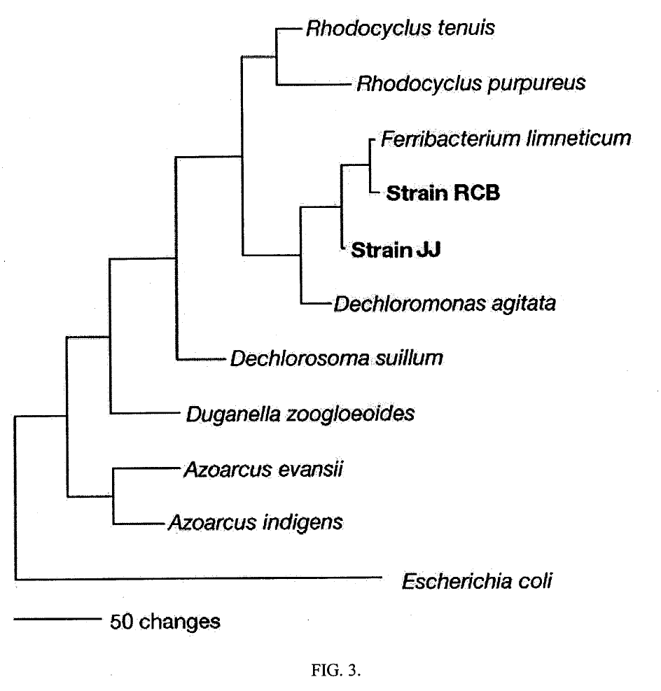

Only those regions sequenced in all of the organisms (1456 nucl.) were used in the subsequent phylogenetic analyses. Distance, parsimony, and maximum likelihood analysis of the aligned sequences was performed using PAUP* 4.0. Bootstrap analysis was conducted on 100 replications using a heuristic search strategy to assess the confidence level of various clades. Genbank accession numbers for sequences represented in FIG. 3 are: Rhodocyclus tenuis (D16209), Rhodocyclus purpureus (M34132), Ferribacterium limneticum (Y17060), Dechloromonas agitata (AF047462), Dechlorosoma suillum (AF170348), Duganella zoogloeoides (previously Zoogloea ramigera, X74913), Azoarcus evansii (X77679), Azoarcus indigens (L15531), and Escherichia coli (J01859).

### Example 2

### Morphology of RCB and JJ

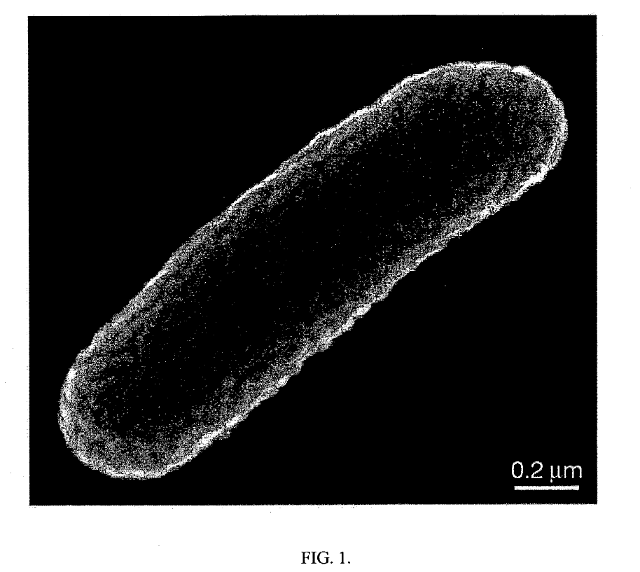

The scanning electron micrograph of anaerobically grown cells of RCB shows that they are rods 1.8 μm by 0.5 μm (FIG. 1).

### Example 3

### Growth of strain RCB and JJ

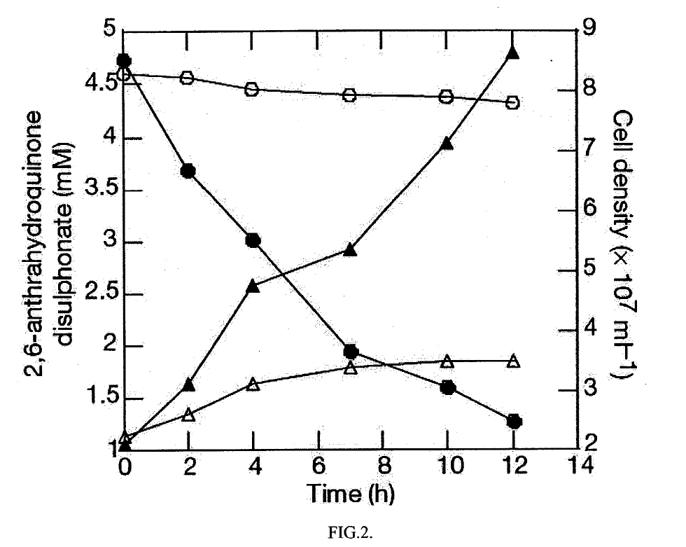

Both strains grew with air or nitrate as alternative electron acceptors. Nitrate is reduced to N₂ gas. Strain RCB also grew with chlorate or perchlorate, completely reducing them to chloride. Both strains RCB and JJ coupled growth to the complete oxidation of various simple organic acids with nitrate as the sole electron acceptor including formate (10 mM), acetate (10 mM), propionate (5 mM), butyrate (5 mM), lactate (10 mM), succinate (5 mM), pyruvate (10 mM), and benzoate (0.5 mM). In addition, both strains also oxidized toluene (1 mM). Both strains grew anaerobically with the reduced humic substances analog 2,6-anthrahydroquinone disulfonate (AHDS) as the electron donor and acetate (0.1 mM) as the carbon source (FIG. 2). Only slight growth was observed if the AHDS was omitted which was probably due to the presence of the small amount of acetate (0.1 mM) added as a carbon source (FIG. 2). No AHDS oxidation occurred in the absence of an electron acceptor.

### Example 4

### Phylogenetic Relation of RCB and JJ

Phylogenetically, strains RCB and JJ are closely related to each other (98.1% 16S rDNA sequence similarity) and both are members of the Dechloromonas subgroup in the β subclass of the Proteobacteria (Achenbach et al., 2001). Their closest relative is Ferribacterium limneticum (FIG. 3) which is an anomaly in this subgroup (Achenbach et al., 2000) as it is a strictly anaerobic Fe(III)-reducer. All other members of the subgroup are facultative anaerobic (per)chlorate-reducers (Achenbach et al., 2000; 2001).

### Example 5

### Anaerobic Oxidation of Benzene by JJ and RCB

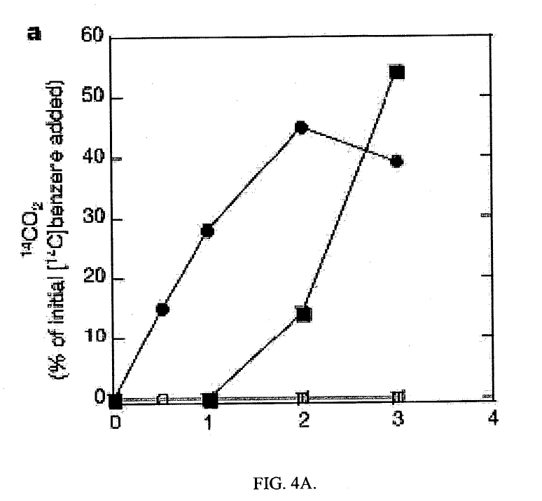

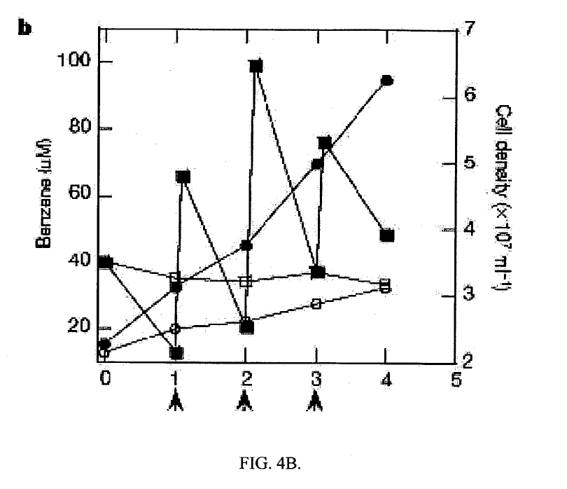

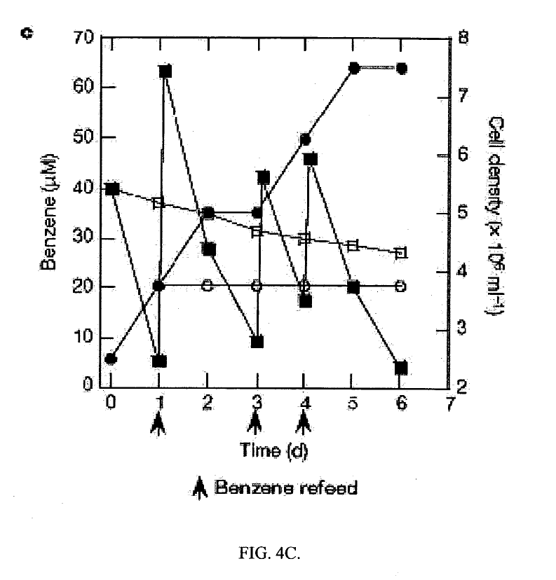

Both strains RCB and JJ oxidized benzene anaerobically with nitrate as electron acceptor (FIG. 4A). [¹⁴C]-labeled benzene was oxidized to ¹⁴CO₂. No [¹⁴C]-benzene oxidation occurred if the electron acceptor was omitted (FIG. 4A). In the case of strain RCB, once ¹⁴CO₂ production ceased, 45% of the original [¹⁴C]-label could be accounted for as ¹⁴co₂. HPLC analysis indicated that 47% of the initial benzene still remained non-degraded in the culture media relative to heat-killed controls. These results account for 92% of the initial [¹⁴C]-label suggesting that benzene is completely oxidized to CO₂. In support of this, HPLC analysis coupled to liquid scintillation counting indicated that the residual [¹⁴C]-label in the aqueous phase was only present as benzene.

In parallel incubations with the aerobic benzene-oxidizer Pseudomonas strain JS-150, ¹⁴CO₂ production was not observed unless O₂ was added (data not shown), demonstrating that benzene oxidation by strains RCB and JJ was anaerobic and was not the result of O₂ contamination. Because of the similar morphology, phylogeny, and physiology of these two strains, strain RCB was selected for a more in-depth characterization of anaerobic benzene oxidation coupled to nitrate reduction.

With nitrate as the sole electron acceptor, the oxidation of 163±19 μM benzene (±standard deviation, n=3) by strain RCB resulted in the reduction of 843±64 μM nitrate (±standard deviation, n=3). This represents greater than 86% of the theoretical ratio for benzene oxidation coupled to nitrate reduction according to: C₆H₆+6NO₃⁻6H⁺→6CO₂+3N₂+6H₂O

In an active benzene-degrading culture of strain RCB with nitrate as the electron acceptor, cell number increase was concomitant with benzene disappearance (FIG. 4B). Minimal growth occurred in the absence of benzene (FIG. 4B) and was probably the result of carryover from the inoculum. The small amount of benzene lost in the heat-killed controls was due to absorption into the butyl rubber stoppers. Previous studies have demonstrated similar losses of benzene into butyl rubber stoppers due to absorption Grbic-Galic et al., 1987; Coates et al., 1996). When the active culture from the growth curve shown in FIG. 4B was used to inoculate fresh media, benzene was again rapidly degraded and cell increase was concomitant with benzene removal (FIG. 4C) demonstrating that strain RCB could grow and be transferred in media with benzene as the sole electron donor and nitrate as the sole electron acceptor. When strain RCB was grown anaerobically with nitrate and UL-[¹⁴C]-benzene, 2.0% of the [¹⁴C]-label was associated with the retentate after the culture was filtered through a 0.2 μm pore size filter. A similar filtration of a heat-killed control retained 0.8% of the [¹⁴C]-label suggesting that 1.2% of the [¹⁴C]-label was incorporated into biomass. In contrast, 3.0% of the [¹⁴C]-label was incorporated into the biomass in an aerobically grown culture. These low values of incorporation of [¹⁴C]-label into biomass are similar to values reported previously for a nitrate-reducing benzene-oxidizing enrichment culture (5-8%) (Burland et al, 1999) and are supportive of the low cell yield for anaerobic growth on benzene observed for strain RCB in FIG. 4B.

### Example 6

### Degradation of Benzene in Sediment Samples

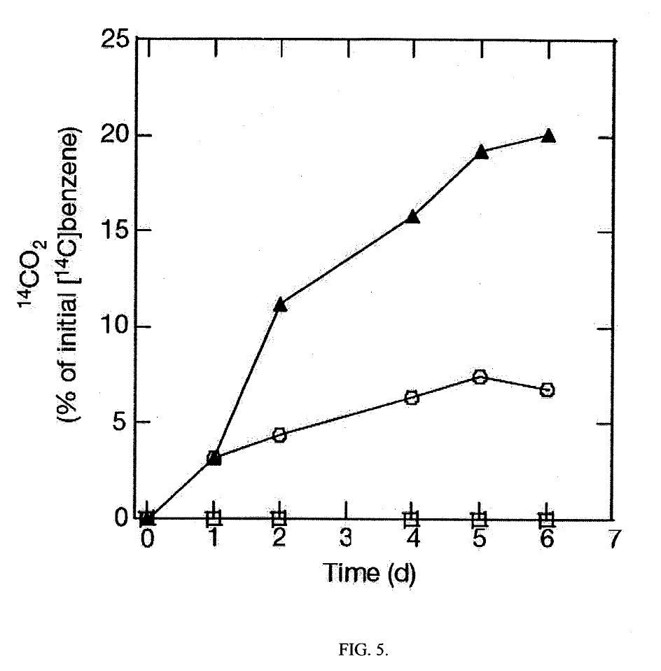

In order to determine the applicability of these organisms to the attenuation of benzene in the natural environment, anoxic aquatic sediments amended with [¹⁴C]-benzene and nitrate were inoculated with an active culture of strain RCB. Geochemical analysis of the sediment revealed that the indigenous nitrate and sulfate concentrations were 22 μM and 867 μM respectively. No chlorate was present. Total iron content was 38 mM and 45% of this (17 mM) was present as Fe (II). Determination of the terminal electron-accepting process in these sediments using [¹⁴C]-acetate (Coates et al., 1996) indicated that Fe(III) reduction was predominant (data not shown). ¹⁴CO₂ from UL-[¹⁴C]-benzene was rapidly evolved from samples amended with both strain RCB and nitrate (FIG. 5). Interestingly, benzene oxidation also occurred in the unamended sediments (FIG. 5). This was unlikely to be due to O₂ contamination, as the high concentrations of Fe (II) in the sediment would rapidly remove O₂. It is also interesting to note that the indigenous benzene oxidation was inhibited by the addition of nitrate when strain RCB was omitted (FIG. 5). Similar inhibition by nitrate addition has previously been reported for a benzene-oxidizing sulfate-reducing sediment (Anderson et al., 2000).

### Example 7

### Re-Initialization of Benzene Oxidation

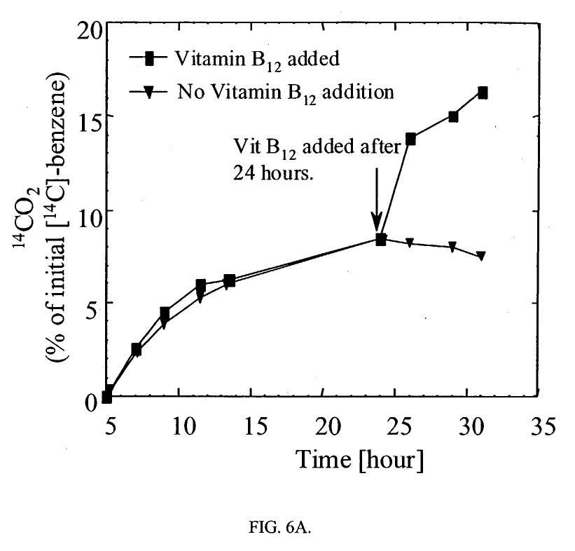

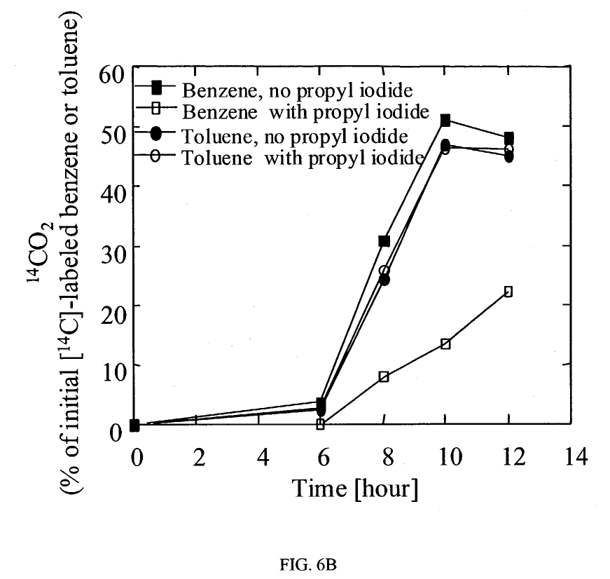

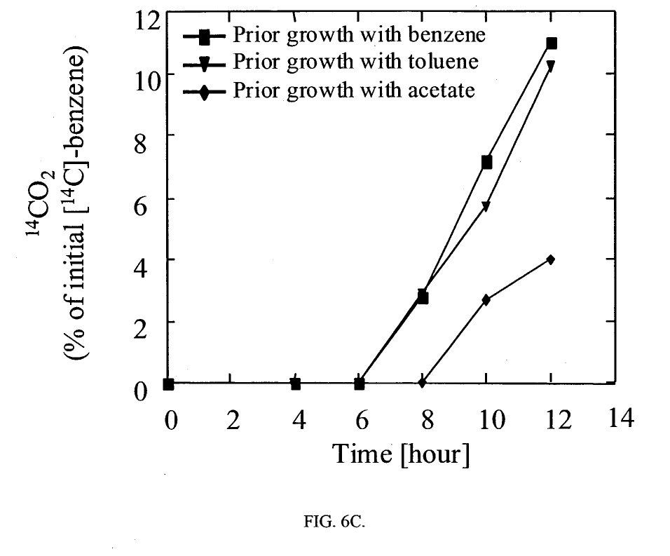

A concentration of 1 mg/liter of vitamin B12 was added to a washed whole cell suspension of RCB once benzene oxidation had ceased. This resulted in a rapid re-initialization of benzene oxidation (FIG. 6A). In contrast, no further benzene oxidation occurred in the unamended control culture (FIG. 6A). This result suggested that methylation may be integral to anaerobic benzene oxidation by strain RCB. In support of this, the addition of propyl iodide, a specific inhibitor of cobalamine-mediated reactions, inhibited anaerobic benzene oxidation by RCB (FIG. 6B). Similar experiments performed with toluene showed that the propyl iodide had no effect on aerobic toluene oxidation by strain RCB (FIG. 6B). Benzene oxidation was significantly enhanced if the washed cell suspension was prepared from cultures grown in the presence of benzene (FIG. 6C) indicating that the enzymes required for benzene oxidation were induced by the presence of benzene. The same was observed when cells were grown in the presence of toluene. But the addition of potential intermediates of benzene degradation such as toluene, benzoate, benzaldehyde, benzylalcohol, or acetate to anoxic washed whole-cell suspensions inhibited benzene oxidation by strain RCB (data not shown). In contrast, similar additions of catechol, phenol, cyclohexane, benzylsuccinate, or fumarate had no significant effect (data not shown).

### Example 8

### Field Trial

To perform a field demonstration of a bioremediative technology for BTEX contamination based on the novel organism Dechloromonas strain RCB, a field trial was undertaken at a contaminated site. The site was contaminated as a result of a leaking underground storage tank used for petroleum storage during the operation of a gas station. The average benzene concentration in the soil was 2830 parts per billion. The soil pH was 6.8, and the nitrate and sulfate contents were 24 μM and 809 μM respectively.

Two plots, a treatment plot and a control plot were excavated side by side separated by a 1 m clay wall. The plot dimensions were 4.5 m by 4.5 m by 3 m deep. The clay wall was draped with polyethylene plastic to act as a hydraulic barrier between the two plots. The soil excavated from the two plots was mixed and homogenized in a farm muck spreader. The soil was then used to back fill the two plots in 1 ft intervals. At each interval, the soil in the treated plot was amended with active cells of Dechloromonas strain RCB to give a final cell density of 1.44×10⁷ cells/g of soil. In addition, the treated plot was also amended with nitrate to give a final concentration of 18.6 parts per million (300 μM). The control plot was amended with an equivalent volume of ground water (150 gal.) collected outside of the contamination plume.

Core samples were collected from the plot sites prior to excavation, immediately after back filling, and on a subsequent regular basis thereafter. The collected core samples were sealed and immediately transported back to the laboratory for hydrocarbon and nitrate analysis. Analyses were performed as previously outlined for the lab trial using gas chromatography with flame ionization detection.

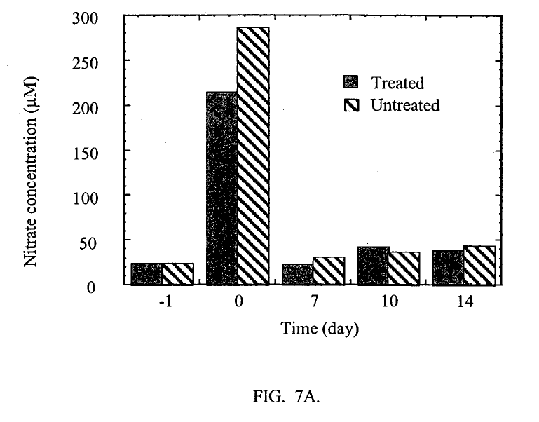

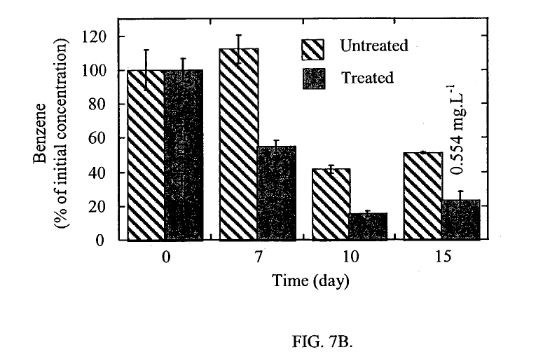

Nitrate analysis of the core samples collected from both plots indicated that the nitrate concentration was significantly elevated as a result of nitrate amendment to the treated plot (FIG. 7A). As nitrate was only amended to the treated plot, this result indicates that the plastic barrier between the plots was ineffective and plots were not hydraulically isolated from each other. The rapid decrease in nitrate content in both plots between day 0 and day 7 (FIG. 7A) was probably the result of both microbial utilization and hydraulic dilution by inflowing groundwater.

As expected, the benzene content of the soil at day 0 (2418 ppb) was significantly lower than prior to excavation, treating, and backfilling (2830 ppb). This was probably the result of evaporation and aerobic microbial degradation of the benzene as the soil was exposed to oxygen during the processing. Benzene analysis over the subsequent 15 days of the trial indicated that the benzene concentration rapidly decreased in the treated plot with an 80% removal achieved within 10 days. As expected, the benzene content of the untreated plot also decreased by 55% during the trial period, although not to the same extent as the treated side (FIG. 7B). The observed benzene degradation in the untreated side probably results from aerobic microbial degradation of the benzene stimulated by exposure to oxygen during soil processing.

The results outlined above indicate that the treatment of the benzene contaminated soils at the field trial site shows great promise. The application of the Dechloromonas strain RCB-based technology showed a significant difference with an untreated control plot and resulted in a rapid removal of 80% of the original benzene content of the soil within 10 days.

### Example 9

### Lab Trials

To perform a lab demonstration of a bioremediative technology for BTEX contamination based on the novel organism Dechloromonas strain RCB, a lab trial was undertaken using soil samples collected from a contaminated site. Three ten foot core samples were collected from heavily contaminated, medium contaminated, and lightly contaminated sections of the site using a Geoprobe. All core samples were pooled and mixed in a plastic bucket prior to transportation back to the laboratory. The pH of the site was checked with a portable electronic pH probe.

Nitrate and sulfate concentrations in soil samples were analyzed by ion chromatography (IC) with chemical suppression and conductivity detection using a Dionex AS-9SC column with a carbonate/bicarbonate mobile phase at a flow rate of 2 ml per min. Soil samples (2 g) were suspended in 4 mL of distilled water and vortexed for 1 minute. The vortex samples were filtered through 0.2 μm filters and injected into the IC. Results were compared against suitable standards and nitrate/sulfate concentrations were calculated.

Bacterial genomic DNA from soil samples were obtained using the FastDNA SPIN Kit for Soil (BIO 101, Inc.). Genomic DNA was used with primers that were specific for bacterial 16S ribosomal DNA or that were specific for signature sequences within the 16S rDNA of Dechloromonas strain RCB-type organisms. These primers were used in 50-μl PCR mixtures that contained 36 μl double-processed tissue culture water (Sigma), 1.5 mM MgCl₂, Mg-free thermophilic DNA polymerase, lx buffer (Promega), each deoxynucleoside triphosphate at a concentration of 0.2 mM, 62.5 ng of each primer, 0.25 μl of Taq polymerase, and 1 μl of genomic DNA suspension. Amplification was performed by using a Perkin Elmer GeneAmp PCR System 2400 under the following conditions: 94° C. for 3 min, followed by 30 cycles consisting of 94° C. for 1 min, 55° C. for 1 min, 72° C. for 1 min, and a final step consisting of 10 min at 72° C. Amplified PCR products were run in a 0.7% low EEO grade electrophoresis gel made with 1× TAE buffer.

Trial A. Approximately 500 g of the collected combined soil samples were added to each of 28 mason jars. The jars were amended (with a final volume of 50 ml) as outlined in Table 1. The treatments were designed to determine the optimum conditions for benzene degradation by Dechloromonas strain RCB in the soil samples. The treated samples were divided so that two jars of each treatment (14 jars) were incubated in the dark at room temperature in an anaerobic chamber under a nitrogen headspace. The second set of jars were also incubated in the dark but were left exposed to an oxic atmosphere. Subsamples (1 g) were collected at regular intervals for enumeration of Dechloromonas strain RCB and 2 g subsamples were collected for benzene analysis. Enumeration studies for strain RCB were performed using a selective medium and a pour plate technique.

TABLE 1
Treatment scheme of collected samples for Trial A

| Treatment | Water | Nitrate | Strain RCB | ^{a}Phosphate pH |
|---|---|---|---|---|
| number | (ml) | (mM) | (cells/g) | buffer nutrient mix |
| 1-4 | 50 | 0 | 0 | 0 |
| 5-8 | 50 | 5 | 0 | 0 |
| 9-12 | 50 | 0 | 1 × 10⁶ | 0 |
| 13-16 | 50 | 5 | 1 × 10⁶ | 0 |
| 17-20 | 0 | 5 | 0 | 50 |
| 20-24 | 0 | 0 | 1 × 10⁶ | 50 |
| 25-28 | 0 | 5 | 1 × 10⁶ | 50 |

^{a}The buffer pH was pH 7.0 and it contained (grams per liter) - KH₂PO₄ (3.85), K₂HPO₄ (6.15), NH₄Cl (0.25). In addition 10 ml per liter each of the defined vitamin and nutrient mineral solutions outlined in table 2 were added to the buffer.

TABLE 2
Vitamin and mineral nutrient solutions added to the phosphate pH
buffer for Trial A

| Vitamin Solution | mg/L | Mineral Solution | G/l |
|---|---|---|---|
| Biotin | 2 | NTA | 1.5 |
| Folic acid | 2 | MgSO₄ | 3 |
| Pyridoxine HCl | 10 | MnSO₄.H₂O | 0.5 |
| Riboflavin | 5 | NaCl | 1 |
| Thiamin | 5 | FeSO₄.7H₂O | 0.1 |
| Nicotinic acid | 5 | CaCl₂.2H₂O | 0.1 |
| Pantothenic acid | 5 | CoCl₂.6H₂O | 0.1 |
| B-12 | 0.1 | ZnCl | 0.13 |
| p-aminobenzoic acid | 5 | CuSO₄ | 0.01 |
| Thioctic acid | 5 | AlK(SO₄)₂.12H₂O | |
| | | H₃BO₂ | 0.01 |
| | | Na₂MoO₄ | 0.025 |
| | | NiCl₂.6H₂O | 0.024 |
| | | Na₂WO₄.2H₂O | 0.025 |

Anoxic R2A agar amended with 10 mM acetate and 10 mM chlorate was used as the selective medium. Collected subsamples were serially diluted in anaerobic saline buffer down to 10⁻⁹. Triplicate subsamples (1.0 ml) from each of the individual serial dilution tubes were used to prepare pour plates. The plates were incubated in anaerobic gas pack jars at 30° C. Colony forming units (CFU) on the plates were counted after 48 hours and the results were compared with those obtained from unamended soils to account for an indigenous population of chlorate-reducing bacteria.

Soil subsamples collected for benzene analysis were placed into 10 ml serum bottles which were subsequently sealed with thick butyl rubber stoppers and placed in a 65° C. water bath for 1 hour. After 1 hour 0.1 ml headspace samples were collected by syringe and analyzed for benzene content by gas chromatography with FID detection. The results were compared against known standards.

The pH of the soil was determined to be circumneutral at pH 6.8. Ion chromatography of a combined soil sample revealed that nitrate concentrations were low and averaged 1.4 ppm (24 μM) while sulfate concentrations were relatively high at 77.7 ppm (809 μM). Molecular analysis of soil samples revealed that there were no indigenous populations of the Dechloromonas bacterial species in the soils collected from the site.

As outlined above, the soil samples did not contain an indigenous population of the benzene degrading Dechloromonas strain RCB. The soils were subsequently inoculated from an active culture of strain RCB to obtain a final cell concentration of 1×10⁶ cells g⁻¹ soil. Interestingly, although Dechloromonas type organisms were not present naturally in the soil, there was an indigenous population of chlorate reducing bacteria (Table 3). Unexpectedly, the cell numbers of the inoculated soils were 1 to 2 orders of magnitude lower (1-2×10⁴) than calculated and were not significantly higher than the uninoculated soil (Table 3). This would imply that either the active cell count in the inoculum was lower than expected or, alternatively, the organism, Dechloromonas strain RCB, did not survive the inoculation step.

TABLE 3
Results of microbial enumeration studies for Trial A

| | |
|---|---|
| Aerobic | Anaerobic |

| Treatment | Day 0 | Day 8 | Day 0 | Day 8 |
|---|---|---|---|---|
| Soil + Distilled Water | 1.3 × 10⁴ | 0 | 1.5 × 10⁴ | 1.3 × 10⁴ |
| (DW) | | | | |
| Soil + Nitrate in DW | 1.2 × 10⁴ | 1.4 × 10⁵ | 1.0 × 10⁴ | 2.3 × 10⁴ |
| Soil + strain RCB in DW | 2.1 × 10⁴ | 2.0 × 10⁴ | 1.6 × 10⁴ | 1.8 × 10⁴ |
| Soil + strain RCB nitrate | 2.2 × 10⁴ | 2.0 × 10⁵ | 9.3 × 10⁴ | 1.2 × 10⁵ |
| in DW | | | | |
| Soil + strain RCB, nitrate | 4.0 × 10³ | 1.0 × 10⁴ | 1.6 × 10⁴ | 8.0 × 10⁴ |
| in buffer | | | | |

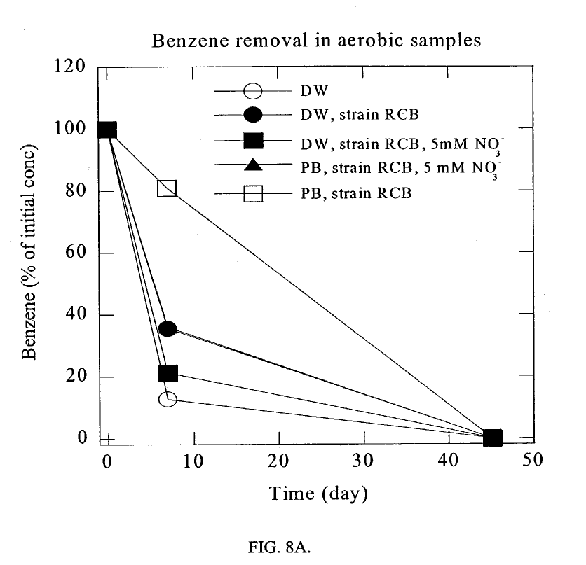

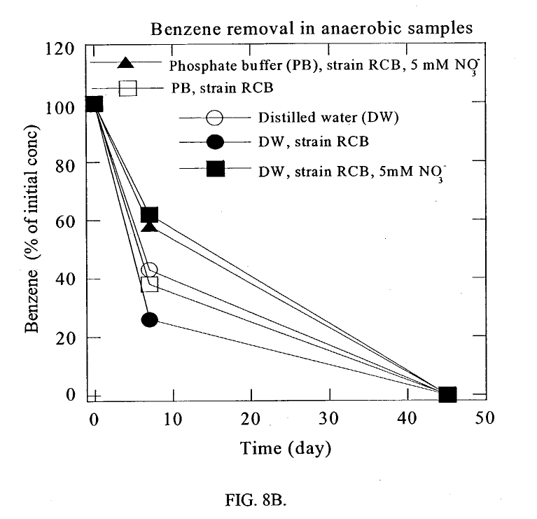

Benzene analysis indicated that the average benzene concentration in the soil samples was 743 ppb. Interestingly, the benzene concentration decreased in all samples regardless of the treatment regime (Table 4) and after 45 days benzene concentrations in all soil samples was below detection (<0.1 ppb) (FIGS. 8A and 8B). As the inoculated Dechloromonas population in these samples was significantly lower than the expected value (Table 3), the observed benzene removal was probably mediated by the indigenous benzene degrading aerobic organisms which were stimulated by the introduction of oxygen into the samples during processing.

TABLE 4
Results of benzene removal studies for Trial A

| | |
|---|---|
| Aerobic | Anaerobic |

| | Day 0 | Day 7 | Day 0 | Day 7 |
|---|---|---|---|---|
| Treatment | (ppb) | (ppb) | (ppb) | (ppb) |
| Soil + Distilled Water (DW) | 678 | 86 | 743 | 323 |
| Soil + Nitrate in DW | 630 | 334 | 1134 | 587 |
| Soil + RCB in DW | 445 | 158 | 796 | 205 |
| Soil + RCB nitrate in DW | 638 | 135 | 792 | 489 |
| Soil + RCB, nitrate in buffer | 565 | 459 | 705 | 266 |

Trial B. Trial B was prepared as a repetition of Trial A and to focus in on the effectiveness of strain RCB on benzene removal from the site samples under anaerobic conditions. The soil samples (10 g) were dispensed into 30 ml serum bottles and sealed with thick butyl rubber stoppers under an N₂ gaseous phase. Amendments to each bottle were made from anoxic sterile stock solutions to give the treatments outlined in Table 5. Because of the problems associated with the inoculum in Trial A, a fresh culture was prepared and used to inoculate the soil to give a final cell density of 1×10⁸ cells g⁻¹ soil. All samples were incubated under a headspace of N₂. As the soil samples were circum neutral pH phosphate buffer was not used in this experiment. The effect of the addition of acetate (5 mM) as an additional carbon/energy source for the organisms was also tested, as was the addition of bran (0.5 g) as a support matrix (Table 5). In this experiment only benzene removal was followed with time. All samples were prepared in duplicate.

TABLE 5
Treatment scheme of collected samples for Trial B

| Treatment | Water | Nitrate | Strain RCB | Acetate | Bran |
|---|---|---|---|---|---|
| number | (ml) | (mM) | (cells/g) | (mM) | (g) |
| 1 | 1.5 | 0 | 0 | 0 | 0 |
| 2 | 1.5 | 5 | 1 × 10⁷ | 0 | 0 |
| 3 | 1.5 | 5 | 1 × 10⁷ | 5 | 0 |
| 4 | 1.5 | 5 | 1 × 10⁷ | 5 | 0.5 |

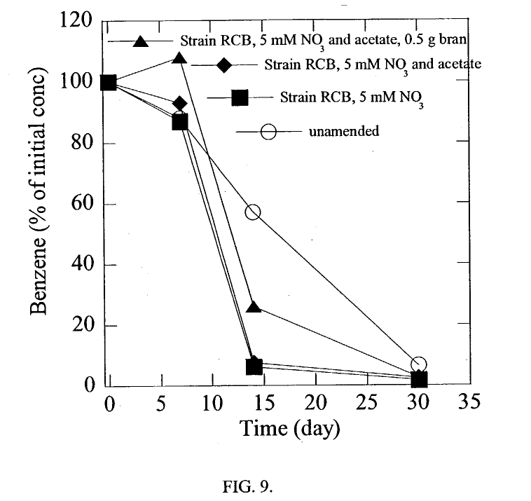

As in Trial A above, benzene concentrations in all samples reduced with time (Table 6, FIG. 9). Benzene removal was most rapid in samples amended with strain RCB and nitrate, and after 30 days incubation benzene concentrations were reduced to 16 ppb, which was an order of magnitude less than the unamended samples (Table 6). Benzene removal in samples amended with acetate was initially slow as expected because the acetate was preferentially utilized; however, after 7 days benzene removal occurred rapidly, and benzene concentrations dropped to 21 and 36 ppb in the acetate and acetate and bran amended samples, respectively.

TABLE 6
Results of benzene removal studies for Trial B

| | Day 0 | Day 7 | Day 14 | Day 30 |
|---|---|---|---|---|
| Treatment | (ppb) | (ppb) | (ppb) | (ppb) |
| Unamended | 2028 | 1787 | 1157 | 133 |
| Strain RCB, nitrate | 943 | 822 | 58 | 16 |
| Strain RCB, nitrate, acetate | 940 | 873 | 71 | 21 |
| Strain RCB, nitrate, acetate, bran | 1380 | 1476 | 356 | 36 |

Conclusions. The results of the lab trials indicate that Dechloromonas strain RCB is not indigenous to the soils at the contaminated site. However, benzene removal can still be stimulated in these soils. The enumeration studies of Trial A suggest that Dechloromonas strain RCB did not survive the inoculation; however, benzene removal did occur and was probably mediated by the indigenous microbial population in the soil samples which were stimulated by exposure to oxygen during the sample transportation and processing. The addition of a pH buffer appears to be unnecessary and may in fact be inhibitory. Trial B indicated that although benzene was again degraded by the indigenous microbial populations, the rate of degradation was significantly enhanced by the addition of an active culture of Dechloromonas strain RCB. In the presence of strain RCB, benzene removal occurred within 15 days relative to more than 30 days in the absence of RCB.

### Example 10

### Materials and Methods for Example 11

Media and stock preparation. All media and solutions were prepared using strict anaerobic techniques as previously described (Coates et al., 2001a; Lloyd et al., 2001; Heider et al., 1997). All culturing was done in sterile sealed serum bottles with N₂—CO₂ (80-20, vol/vol) in the headspace at 30° C. Sterile anoxic aqueous stock solutions of propyl iodide (1 mM), vitamin B₁₂ (1 gm L-1) toluene (3.5 mM), benzaldehyde (1 mM), benzylalcohol (1 mM), nitrate (1M) were prepared and stored at 4° C. in the dark. Amendments to the respective experimental samples were made using sterile N₂-flushed syringes as required.

[¹⁴C]-label experiments. [¹⁴C]-Benzene (1 μCi) was added to fresh anoxic basal media containing 5 mM nitrate and was inoculated (10% v/v) with an active culture of Dechloromonas strain RCB. Potential intermediates benzaldehyde (50 μM), benzylalcohol (50 μM), and toluene (35 μM) were amended as necessary from anoxic aqueous stock solutions to the experimental samples. The production of ¹⁴CO₂ was monitored as previously described by analysis of 1 ml headspace samples using gas chromatography with gas proportional counting detection (GC-GPC) (Coates et al., 2001 a).

Permeabilized cell experiments. Active cells of strain RCB were premeabilized by the addition of Triton X-100 to a previously prepared anoxic, washed cell suspension. The washed cell suspension was prepared in phosphate buffer (10 mM, pH 7) as previously described (Bruce et al., 1999) using an active culture of strain RCB grown in anoxic basal media with 16 mM nitrate and 10 mM acetate. Aliquots (0.1 ml) of the cell suspension were transferred into degassed vials containing 3 ml phosphate buffer. Triton X-100 (1% final concentration, v/v) was added to permeabilize the cells. [¹⁴C]-benzene (2 μCi), vitamin B₁₂ (0.25 g L⁻¹) and nitrate 10 mM) were added to experimental vials from anoxic aqueous stock solutions. All vials were incubated at 30° C. When 20% of the [¹⁴C]-benzene was oxidized to ¹⁴CO₂, the respective vials were further amended with toluene, benzaldehyde, or benzylalcohol (50 μM each) as “cold traps”, and the vials were incubated for a further four hours.

Analytical techniques. ¹⁴CO₂ production in the headspace of cultures amended with [¹⁴C]-labeled benzene or toluene was determined by gas chromatography with gas proportional counting detection (GC-GPC) as previously described (Coates et al., 2001 a). In addition, gaseous phase [¹⁴C]-benzene and [¹⁴C]-toluene were also analyzed in 1 ml headspace samples by the GC-GPC using an 80/100 Carbopack C/0.1% SP-1000 stainless steel column (Supelco) and an N₂ carrier gas. Concentrations of aqueous-phase [¹⁴C]-benzene and other potential [¹⁴C]-catabolic intermediates were determined by HPLC using a Supelcosil LC-18 column (column: 25632-06) and acetonitrile (60%)-water (40%) mobile phase. [¹⁴C]-labeled compounds were detected by a flow through radioisotope detector (β-RAM model 3, IN/US Systems). Prior to sampling for aqueous phase analysis, samples were amended with 500 μl of 6N HCl to remove all dissolved ¹⁴CO₂. The samples were then filtered through a 0.2 μm filter and injected directly into the HPLC for analysis.

### Example 11

### Activation of Benzene for Anaerobic Microbial Catabolism

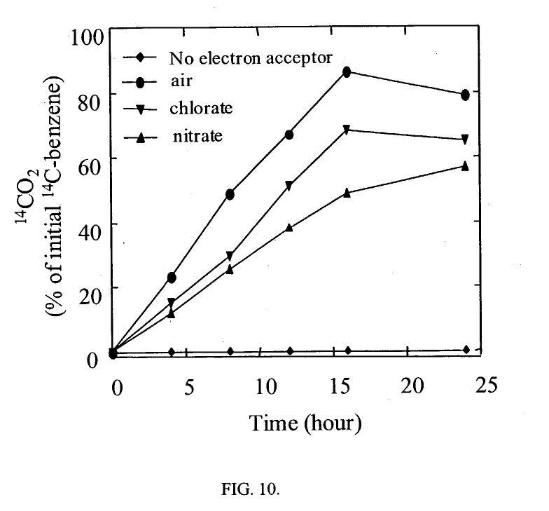

To identify the mechanism of activation of benzene for anaerobic microbial catabolism, benzene oxidation was coupled to the reduction of either nitrate or perchlorate by Dechloromonas strain RCB. [¹⁴C]-Labeled benzene was oxidized to ¹⁴CO₂ (FIG. 10). Concentrations of benzene as high as 160 μM were removed within five days and growth was concomitant with loss of benzene (Coates et al., 2001a). Benzene catabolism by strain RCB was significantly enhanced if a washed whole-cell suspension was prepared from cultures previously grown with either benzene or toluene (FIG. 6C). The induction by toluene is in contrast to previous studies which suggested that the enzymes involved in the conversion of aromatic compounds to central intermediates, for example benzyol-CoA, are very specific and are only induced by the substrate of the pathway and not by structural analogs. Furthermore, strain RCB simultaneously oxidized both toluene and benzene with nitrate as the sole electron acceptor (data not shown).

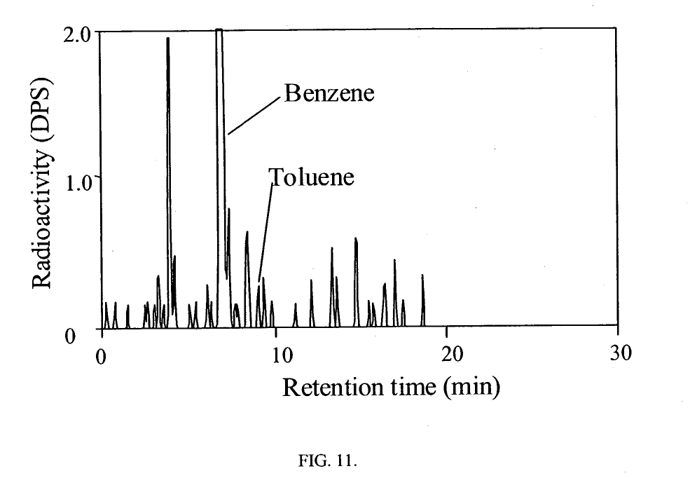

Gas chromatographic analysis of the headspace of [¹⁴C]-benzene-metabolizing permeabilized cells of strain RCB amended with unlabeled toluene indicated the presence of two radioactive peaks in addition to [¹⁴C]-benzene after 12 hours incubation. Comparison of retention time of these peaks with known standards identified them as ¹⁴CO₂ and [¹⁴C]-toluene. A similar peak was not observed in non-active control cultures from which the electron acceptor was omitted indicating that the [¹⁴C]-toluene was formed as a result of [¹⁴C]-benzene catabolism and was not a contaminant of the [¹⁴C]-benzene stock or the result of an abiotic interaction between the [¹⁴C]-benzene and the non-labeled toluene in the medium. GC analysis of ether extracts of the culture broth similarly showed the presence of [¹⁴C]-toluene peak (data not shown) which accounted for 2.02% of the total [¹⁴C]-label. Again, no [¹⁴C]-toluene was observed in the non-active control cultures. Furthermore, HPLC analysis of the cell-free broth from the same cultures also revealed the presence of a radioactive peak with a retention time identical to a [¹⁴C]-toluene standard (FIG. 11). This peak accounted for 1.06% of the total [¹⁴C]-label content of the sample and again was only apparent in benzene-metabolizing cultures (data not shown). The difference in the relative amounts of [¹⁴C]-labeled toluene using the two different forms of chromatography was expected as gas chromatography only detects volatile components in the samples, whereas liquid chromatography will detect all aqueous soluble components. In support of this, the HPLC chromatograms contained 15 unidentified radioactive peaks in addition to the toluene and benzene (FIG. 11), whereas no additional peaks apart from ¹⁴CO₂ were observed in the GC chromatograms (data not shown).

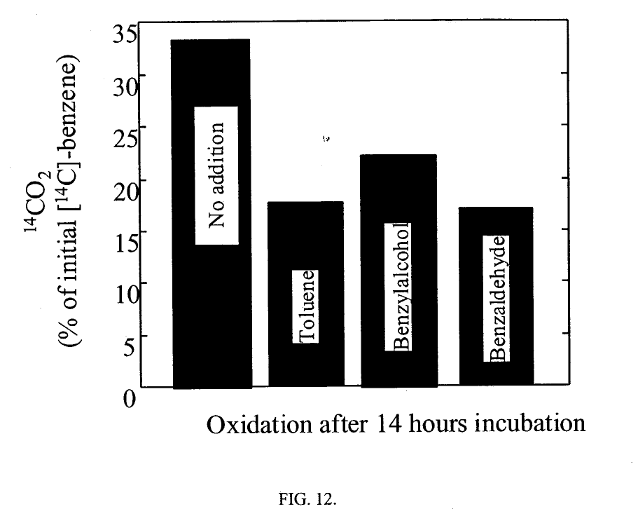

These results suggest that benzene may be activated for anaerobic catabolism by biomethylation to form toluene by strain RCB. In support of this, the rate of ¹⁴CO₂ production from [¹⁴C]-benzene by a growth culture of strain RCB was significantly depressed by the addition of unlabeled toluene to the medium (FIG. 12). Conversely, the rate of ¹⁴CO₂ production from [¹⁴C]-toluene was unaffected by the addition of unlabeled benzene (data not shown). Previous studies have demonstrated that the addition of an unlabeled form of a catabolic intermediate (toluene) to an active culture may cause a reduction in the rate of ¹⁴CO₂ production from a [¹⁴C]-labeled substrate ([¹⁴C]-benzene) (Edwards et al., 1994; Lovley et al., 1995). This is because the biogenic labeled form of the transient intermediate ([¹⁴C]-toluene) is exchanged into the unlabeled pool and the associated [¹⁴C]-label does not get further metabolized. As such, the production of ¹⁴CO₂ would be expected to be lower in the presence of toluene if toluene was an intermediate of the pathway. ¹⁴CO₂ production from [¹⁴C]-benzene by strain RCB was also inhibited by the addition of benzylalcohol or benzaldehyde (FIG. 12). These compounds are known competitive inhibitors of benzylsuccinate synthase (BSS), an enzyme which mediates the addition of fumarate to the methyl group of toluene to form benzylsuccinate during anaerobic toluene degradation by several organisms (Heider et al., 1997; Biegert et al., 1995; Biegert et al., 1996; Leuthner et al., 1998).

The inventors considered that if further catabolism of the biogenically formed [¹⁴C]-toluene by strain RCB was repressed by the inhibition of the BSS then its presence should be detectable in a treated cultures. In support of this, GC analysis of the headspace of cultures amended with either benzylalcohol or benzaldehyde revealed the presence of [¹⁴C]-toluene in addition to [¹⁴C]-benzene and 1⁴CO₂. The [¹⁴C]-toluene accounted for 0.2% and 0.1% of the total [¹⁴C]-label for the benzylalcohol and benzaldehyde treated cultures respectively. In contrast, [¹⁴C]-toluene was not observed in unamended controls.

The results of the present study demonstrate that toluene is formed as an intermediate in the anaerobic nitrate-dependent catabolism of benzene by strain RCB and that biomethylation may be the first step in the pathway. This is the first identification of this important biochemical reaction in a microbial system. Biologically mediated alkylation of benzene to toluene is energetically favorable using either S-adenosyl-methionine (ΔG^{o}=−112.33 kJ/mol) or methyl-tetrahydrofolate (ΔG^{o}=−32.31 kJ/mol) as methyl donors (Coates et al., 2002). Previous microbial studies performed with undefined enrichment and sediment cultures have alternatively indicated that phenol and benzoate were formed as potential intermediates of anaerobic benzene catabolism (Weiner et al., 1998; Caldwell et al., 2000; Phelps et al., 2001; Vogel et al., 1986; Grbic-Galic et al., 1987). Whether these compounds were formed directly from benzene or result from several sequential metabolic steps is unknown. However, in all cases the phenol and benzoate were detected in the culture aqueous phase without the necessity of cell-permeabilization (Weiner et al., 1998; Caldwell et al., 2000; Phelps et al., 2001; Vogel et al., 1986; Grbic-Galic et al., 1987). This suggests that, in contrast to our studies, the benzene was not degraded by a single organism but rather was the result of symbiotic interactions between two or more organisms in these cultures with the production of extracellular intermediates. In support of this conclusion, the majority of these studies were performed using methanogenic enrichments which rely on syntrophic associations of microbial communities for the degradation of organic matter. Furthermore, none of these previous studies were performed under nitrate-reducing conditions which has previously been shown to involve alternative catabolic pathways for aromatic hydrocarbons such as ethylbenzene due to the high redox potential of this electron acceptor (Widdel et al., 2001).

Bioalkylation of benzene to toluene has previously been observed in studies with bone marrow tissue (Flesher et al., 1991) and a similar reaction is proposed to be the first step in the metabolic activation of unsubstituted carcinogenic aromatic hydrocarbons in mammalian systems (Flesher et al., 1990; Flesher et al., 1991b; Stansbury et al., 1994; Lehner et al., 1996). As such, the study described in the present example not only identifies a novel mechanism for the microbial activation of benzene for catabolism in the absence of oxygen but may also offer a good model to gain insight into the carcinogenic activity of several aromatic hydrocarbons in mammalian tissues.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:

Achenbach and Coates, “Disparity between bacterial phylogeny and physiology,” ASM News 66:714-716, 2000.

Achenbach, Bruce, Michaelidou, Fryman, Coates, “Dechloromonas agitata N. N. gen., sp. Nov. and Dechloromonas suillum N. N. gen. Sp. Nov. two novel environmentally dominant (per)-chlorate-reducing bacteria and their phylogenetic position,” Int. J Syst. Evol. Microbiol., 51:527-533, 2001.

Anderson and Lovley, “Anaerobic bioremediation of benzene under sulfate-reducing conditions in a petroleum contaminated aquifer,” Environ. Sci. Techol., 34:2261-2266, 2000.

Anderson and Lovley, “Naphtalene and benzene degradation under Fe(III)-reducing conditions in petroleum-contaminated aquifers,” Bioremed. J, 3:121-135, 1999.

Anderson, Hughes, Veidebaum, Peltonen, Sorsa, “Examination of ras (P21) proteins in plasma from workers exposed to benzene emissions from petrochemical plants and healthy controls,” Mutat Res, 381(2):149-55, 1997.

Anderson, Rooney-Varga, Gaw, Lovley, “Anaerobic benzene oxidation in the Fe(III) reduction zone of petroleum-contaminated aquifers,” Environ. Sci. Technol., 32:1222-1229, 1998.

Berman, Chase, Jr., “Carbon flow in mercury biomethylation by Desulfovibrio desulfuricans,” Appl. Environ. Microbiol., 56:298-300, 1990.

Biegert and Fuchs, “Anaerobic oxidation of toluene (analogues) to benzoate (analogues) by whole cells and by cell extracts of a denitrifying Thauera sp.,” Arch. Microbiol., 163:407-417, 1995.

Biegert, Fuchs, Heider, “Evidence that oxidation of toluene in the denitrifying bacterium Thauera aromatica is initiated by formation of benzylsuccinate from toluene and fumarate,” Eur. J Biochem., 238:661-668, 1996.

Bruce, Achenbach, Coates, “Reduction of (per)chlorate by a novel organism isolated from a paper mill waste,” Environ. Microbiol., 1:319-331, 1999.

Burland and Edwards, “Anaerobic benzene biodegradation linked to nitrate reduction,” Appl. Environ. Microbiol., 65:529-533, 1999.

Caldwell and Suflita, “Detection of phenol and benzoate as intermediates of anaerobic benzene biodegradation under different terminal electron-accepting conditions,” Environ. Sci. Technol., 34:1216-1220, 2000.

Christensen, Kjeldsen, Albrechtzen, Heron, “Attenuation of pollutants in landfill leachate polluted aquifers,” Crit. Rev. Environ. Sci. Techol., 24:119-202, 1994.

Choi and Bartha, “Cobalamin-mediated mercury methylation by Desulfovibrio desulfuricans LS,” Appl. Environ. Microbiol., 59:290-295, 1993.

Choi, Chase Jr., Bartha. “Metabolic pathways leading to mercury methylation in Desulfovibrio desulfuricans LS,” Appl. Environ. Microbiol., 60:4072-4077, 1994.

Coates, Anderson, Lovley, “Anaerobic hydrocarbon degradation in petroleum-contaminated harbor sediments under sulfate-reducing and artifically imposed iron-reducing conditions,” Environ. Sci. Technol., 30:2784-2789, 1996.

Coates, Anderson, Lovley, “Anaerobic oxidation of polycyclic aromatic hydrocarbons under sulfate-reducing conditions,” Appl. Environ. Microbiol., 62:1099-1101, 1996.

Coates, Phillips, Lonergan, Jenter, Lovley, “Isolation of Geobacter species from a variety of sedimentary environments,” Appl. Environ. Microbiol., 62:1531-1536, 1996.

Coates, Woodward, Allen, Philp, Lovley, “Anaerobic degradation of polycyclic aromatic hydrocarbons and alkanes in petroleum-contaminated marine harbor sediments,” Appl. Environ. Microbiol., 63:3589-3593, 1997.

Coates, Bruce, Patrick, Achenbach, “Hydrocarbon bioremediative potential of (per)chlorate-reducing bacteria,” Bioremed. J, 3:323-334, 1999a.

Coates, Michaelidou, Bruce, O'Connor, Crespi, Achenbach, “The ubiquity and diversity of dissimilatory (per)chlorate-reducing bacteria,” App. Environ. Mcirobiol., 65:4234-5241, 1999b.

Coates et al., “Anaerobic benzene oxidation coupled to nitrate reduction in pure culture by two strains of dechloromonas,” Nature, 411:1039-1043, 2001 a.

Coates and Achenbach in Manual of Environmental Microbiology (eds. Hurst, Knudsen, McInerney, Stetzenbach, Walter, ASM Press, Washington, D.C.), 719-727, 2001.

Coates, Chakraborty, McInerney, “Anaerobic benzene biodegradation—a new era,” Res. Microbiol., in press, 2002.

Edwards, E. A., Edwards, A. M. & Grbic-Galic, “A method for detection of aromatic metabolites at very low concentration: application to detection of metabolites of anaerobic toluene degradation,” Appl Environ Microbiol., 60:323-327, 1994.

Fatoki, “Biomethylation in the natural environment: a review,” So. Afr. J Sci., 93:366-370, 1997.

Flesher and Myers, “Bioalkylation of benz[a]anthracene as a biochemical probe for carcinogenic activity. Lack of bioalkylation in a series of six noncarcinogenic polynuclear aromatic hydrocarbons,” Drug Metab. Dispos., 18:163-7, 1990.

Flesher and Myers, “Methyl-substitution of benzene and toluene in preparations of human bone marrow,” Life Sci., 48:843-50, 1991a.

Flesher and Myers, “Rules of molecular geometry for predicting carcinogenic activity of unsubstituted polynuclear aromatic hydrocarbons,” Teratog., Carcinog., Mutagen., 11:41-54, 1991b.

Galushko, Minz, Schink, Widdel, “Anaerobic degradation of naphthalene by a pure culture of a novel type of marine sulfate-reducing bacterium,” Environ. Microbial., 1:415-420, 1999.

Grbic-Galic and Vogel, “Transformation of toluene and benzene by mixed methanogenic cultures,” Appl. Environ. Microbiol., 53:254-260, 1987.

Heider and Fuchs, “Anaerobic metabolism of aromatic compounds,” Eur. J. Biochem., 243:577-596, 1997.

Lehner, Horn, Flescher, “Benzylic carbonium ions as ultimate carcinogens of polynuclear aromatic hydrocarbons,” J. Mol. Structure, 366:203-217, 1996.

Leuthner et al., “Biochemical and genetic characterization of benzylsuccinate synthase from Thauera aromatica: a new glycyl radical enzyme catalysing the first step in anaerobic toluene metabolism,” Mol. Microbiol., 28:615-28, 1998.

Lloyd and Lovley, “Microbial detoxification of metals and radionuclides,” Curr. Opin. Biotechnol., 12:248-253, 2001.

Lovley and Lonergan, “Anaerobic oxidation of toluene, phenol, and p-cresol by the dissimilatory iron-reducing organism, GS-15,” Appl. Environ. Microbiol., 56:1858-1864, 1990.

Lovley, Woodward, Chapelle, “Stimulated anoxic biodegradation of aromatic hydrocarbons using Fe(III) ligands,” Nature, 370:128-131, 1994.

Lovely, Coates, Woodward, Phillips, “Benzene oxidation coupled to sulfate reduction,” Appl. Environ. Microbiol., 61:953-958, 1995.

Lovley, Coates, Blunt-Harris, Phillips, Woodward, “Humic substances as electron acceptors for microbial respiration,” Nature, 382(1):445-448, 1996.

Lovley, “Potential for anaerobic bioremediation of BTEX in petroleum-contaminated aquifers,” J. Ind. Microbiol., 18:75-81, 1997.

Lovley, “Anaerobic benzene degradation,” Biodegradation, 11: 107-116, 2000. Michaelidou, Achenbach, Coates, “Isolation and characterization of two novel (per)chlorate-reducing bacteria from swine waste lagoons,” In Perchlorate in the Environment, Urbansky and Kluwer (Eds.), Academic/Plenum, NY, 271-284, 2000.

Phelps, Kerkhof, Young, “Molecular characterization of a sulfate-reducing consortium which mineralizes benzene,” FEMS Microbiol. Ecol., 27:269-279, 1998.

Phelps, Zhang, Young, “Use of stable isotopes to identify benzoate as a metabolite of benzene degradation in a sulphidogenic consortium,” Environ. Microbiol., 3:600-603, 2001.

Rabus, Nordhaus, Ludwig, Widdel, “Complete oxidation of toluene under strictly anoxic conditions by a new sulfate-reducing bacterium,” Appl. Environ. Microbiol., 59:1444-1451, 1993.

Rabus et al., “Anaerobic initial reaction of n-alkanes in a denitrifying bacterium: evidence for (1-methylpentyl)succinate as initial product and for involvement of an organic radical in n-hexane metabolism,” J. Bacteriol., 183:1707-15, 2001.

Ridgeway, Safarik, Phipps, Carl, Clark, “Identification and catabolic activity of well-derived gasoline-degrading bacteria and a contaminated aquifer,” Appl. Environ. Microbiol., 56:3565-3575, 1990.

Ridley, Dizikes, Wood,. “Biomethylation of toxic elements in the environment,” Science, 197:329-332, 1977.

Stansbury, Flesher, Gupta, “Mechanism of aralkyl-DNA adduct formation from benzo[a]pyrene in vivo,” Chem. Res. Toxicol., 7:254-9, 1994.

Vogel and Grbic-Galic, “Incorporation of oxygen from water into toluene and benzene during anaerobic fermentative transformation,” Appl. Environ. Microbiol., 52:200-202, 1986.

Weiner and Lovley, “Anaerobic benzene degradation in petroleum-contaminated sediments after inoculation with a benzene-oxidizing enrichment,” Appl. Environ. Microbiol., 64:775-778, 1998a.

Weiner and Lovley, “Rapid benzene degradation in methanogenic sediments from a petroleum-contaminated aquifer,” Appl. Environ. Microbiol., 64:1937-1939, 1998b.

Weissbach, Redfield, Dickerman, Brot, “Studies on methionine biosynthesis. effect of alkylcobamide derivatives on the formation of holoenzyme,” J. Biol. Chem., 240:856-62, 1965.

Widdel and Rabus, “Anaerobic biodegradation of saturated and aromatic hydrocarbons,” Curr. Opin. Biotech., 12:259-76, 2001.

Wood and Wolfe, “Propylation and purification of a B12 enzyme involved in methane formation,” Biochemistry, 5:3598-3603, 1966.

What is claimed is:

1. A method for the biodegradation of an aromatic hydrocarbon comprising the steps of:
(a) providing a bacterium of the Dechloromonas species that biodegrades said aromatic hydrocarbon under anaerobic conditions;
(b) contacting an environment containing said aromatic hydrocarbon with said bacterium in the presence of an electron acceptor; and
(c) incubating said bacterium in said environment for a sufficient time to biodegrade said aromatic hydrocarbon.

2. The method of claim 1, wherein said incubation is conducted under aerobic conditions.

3. The method of claim 1, wherein said incubation is conducted under anaerobic conditions.

4. The method of claim 1, wherein said aromatic hydrocarbon comprises benzene, toluene, xylene, or ethylbenzene.

5. The method of claim 1, wherein said environment is substantiated with trace amounts of ferrous iron or acetate.

6. The method of claim 5, wherein said trace amount of ferrous iron is 0.1 mM.

7. The method of claim 5, wherein said trace amount of acetate is 1 mM.

8. The method of claim 1, wherein said electron acceptor comprises oxygen, nitrate, chlorate, perchlorate or manganese.

9. The method of claim 1, wherein said environment comprises sediment, soil, groundwater, industrial waste streams or aquifers

10. The method of claim 1, wherein the bacterium is strain RCB.

11. The method of claim 1, wherein the bacterium is strain JJ.

12. A method for the biodegradation of benzene comprising the steps of:
(a) providing a bacterium that biodegrades benzene under anaerobic conditions;
(b) contacting an environment containing said aromatic hydrocarbon with said bacterium in the presence of an electron acceptor; and
(c) incubating said bacterium in said environment for a sufficient time to biodegrade said aromatic hydrocarbon.

13. The method of claim 12, wherein said incubation is conducted under anaerobic conditions.

14. The method of claim 12, wherein said incubation is conducted under aerobic conditions.

15. The method of claim 12, wherein said electron acceptor comprises oxygen, nitrate, perchlorate, chlorate or manganese.

16. The method of claim 12, wherein said environment comprises sediment, soil, groundwater, industrial waste stream or aquifer.

17. The method of claim 12, wherein said bacterium is of the Dechloromonas species.

18. The method of claim 17, wherein said bacterium is strain RCB.

19. The method of claim 17, wherein said bacterium is strain JJ.

20. An isolated bacterium that biodegrades benzene under anaerobic conditions

21. The bacterium of claim 20, wherein said bacterium is of the Dechloromonas species.

22. The bacterium of claim 21, wherein said bacterium is strain RCB.

23. The bacterium of claim 21, wherein said bacterium is strain JJ.

24. A method for the isolation of a bacterium that biodegrades one or more aromatic hydrocarbons under anaerobic conditions comprising:
(a) obtaining a sample containing (per)chlorate-reducing bacteria;
(b) incubating said bacteria from said sample in an anoxic medium comprising an electron acceptor and an electron donor;
(c) selecting bacteria based on increased cell density in said sample; and
(d) inoculating said selected bacteria onto fresh anoxic medium comprising an electron acceptor and an electron donor,
wherein bacteria growing in step (d) comprise a bacterium that biodegrades aromatic hydrocarbons under anaerobic conditions.

25. The method of claim 24, wherein said steps (c) and (d) are repeated at least once.

26. The method of claim 24, wherein said steps (c) and (d) are repeated at least five times.

27. The method of claim 24, wherein said steps (c) and (d) are repeated at least ten times.

28. The method of claim 24, wherein said electron acceptor comprises (per)chlorate, oxygen, nitrate or manganese.

29. The method of claim 28, wherein said electron acceptor is present at 1 mM-25 mM.

30. The method of claim 28, wherein said chlorate is present at 10 mM.

31. The method of claim 24, wherein said electron donor comprises chlorate, acetate, H₂, volatile fatty acids, aromatic compounds, reduced metals, reduced humic substances or analogs of reduced humic substances.

32. The method of claim 31, wherein said reduced metal is ferrous iron.

33. The method of claim 31, wherein said volatile fatty acids comprise formate, acetate or butyrate.

34. The method of claim 31, wherein said aromatic compounds comprise 4-chlorobenzoate, benzoate, benzene, toluene, ethylbenzene or xylene.

35. The method of claim 31 wherein said analog of reduced humic substances is 2, 6-anthrahydroquinone disulphonate.

36. The method of claim 31, wherein said electron donor is present at 0.08 mM-20 mM.

37. The method of claim 34, wherein said chlorobenzoate is present at 0.5 mM.

38. The method of claim 24, wherein said bacterium degrades one or more aromatic hydrocarbon selected from the group consisting of benzene, xylene, ethylbenzene, hexadecane, napthalene or toluene.

39. The method of claim 24, further comprising subjecting said bacterium to an anoxic agar dilution series.

40. The method of claim 24, wherein steps (b) and (c) are performed at 20-40° C.

41. The method of claim 40, wherein said steps (b) and (c) are performed at 30° C.

42. An isolated bacterium that biodegrades one or more aromatic hydrocarbons under anaerobic conditions, isolated according to the method comprising:
(a) obtaining a sample containing (per)chlorate-reducing bacteria;
(b) incubating said bacteria from said sample in an anoxic medium comprising an electron acceptor and an electron donor;
(c) selecting bacteria based on increased cell density in said sample; and
(d) inoculating said selected bacteria onto fresh anoxic medium comprising an electron acceptor and an electron donor,
wherein bacteria growing in step (d) comprise a bacterium that biodegrades aromatic hydrocarbons under anaerobic conditions.

43. A method of reusing an isolated bacterium that biodegrades one or more aromatic hydrocarbons under anaerobic conditions comprising the steps of:
(a) obtaining bacterial cells from a media that has been depleted of aromatic hydrocarbons;
(b) adding a cobalt-containing compound to the cells from step (a); and
(c) transferring the cobalt-treated cells to a media containing aromatic hydrocarbons.

44. The method of claim 43, wherein said bacterial cells of step (a) are obtained from said media of step (a) after benzene has been fully depleted.

45. The method of claim 43, wherein said bacterial cells of step (a) are obtained from said media of step (a) after benzene has been partially depleted.

46. The method of claim 45, wherein the cobalt treated cells are transferred into the media of step (a).

47. The method of claim 43, wherein the cobalt treated cells are transferred into a fresh environment containing non-degraded aromatic hydrocarbons.

48. The method of claim 47, wherein said fresh environment comprises sediment, soil, groundwater, industrial waste stream or aquifer.

49. The method of claim 43, wherein said cobalt-containing compound is vitamin B₁₂.

50. The method of claim 49, wherein the concentration of said vitamin B₁₂ is at a concentration of 1 mg/liter.

51. The method of claim 43, wherein said media comprises an electron acceptor.

52. The method of claim 51, wherein said electron acceptor comprises (per)chlorate, oxygen, nitrate or manganese.

53. The method of claim 43, wherein said bacterium is of the Dechloromonas species.

54. The method of claim 53, wherein said bacterium is strain RCB.

55. The method of claim 53, wherein said bacterium is strain JJ.

56. The method of claim 43, wherein said aromatic hydrocarbon is benzene, toluene, xylene, or ethylbenzene.

57. A method of enhancing the efficiency of degradation of aromatic hydrocarbons in a media comprising adding a cobalt-containing compound to the media.

58. The method of claim 57, wherein said cobalt-containing compound is vitamin B₁₂.

59. The method of claim 58, wherein said vitamin B₁₂ is added after the initiation of benzene oxidation.

60. The method of claim 59, wherein said vitamin B₁₂ is added 24 hours after the initiation of benzene oxidation.

61. The method of claim 57, wherein said media comprises sediment, soil, groundwater, industrial waste stream or aquifer.

62. The method of claim 57, wherein said biodegradation is carried out by strains of the Dechloromonas sp.

63. The method of claim 62, wherein said strain is RCB.

64. The method of claim 63, wherein said strain is JJ.
